SEQUENCE LISTING (27 sequences)

SEQ ID NO: 1 - protein, 86 aa, Artificial Sequence
  human derived abrogen
KTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 2 - DNA, 258 bp, Artificial Sequence
  human derived abrogen
aaaacctgctatgaggggaatggtcacttttaccgaggaaaggccagcactgacaccatgggccggccctgcctgccctggaactctgccactgtccttcagcaaacgtaccatgcccacagatctaatgctcttcagctgggcctggggaaacataattactgcaggaacccagacaaccggaggcgaccctggtgctatgtgcaggtgggcctaaagccgcttgtccaagagtgcatggtgcatgactgcgcagat

SEQ ID NO: 3 - protein, 86 aa, Artificial Sequence
  mouse derived abrogen
KTCYHGNGDSYRGKANTDTKGRPCLAWNAPAVLQKPYNAHRPDAISLGLGKHNYCRNPDNQKRPWCYVQIGLRQFVQECMVHDCSL

SEQ ID NO: 4 - DNA, 258 bp, Artificial Sequence
  mouse derived abrogen
aaaacctgctatcatggaaatggtgactcttaccgaggaaaggccaacactgataccaaaggtcggccctgcctggcctggaatgcgcctgctgtccttcagaaaccctacaatgcccacagacctgatgctattagcctaggcctggggaaacacaattactgcaggaaccctgacaaccagaagcgaccctggtgctatgtgcagattggcctaaggcagtttgtccaagaatgcatggtgcatgactgctctctt

SEQ ID NO: 5 - protein, 86 aa, Artificial Sequence
  human derived abrogen
KTCYEGNGHFYRGKASTDTMGRPCLPWNSATVLQQTYHAHRSDALQLGLGKHNYCRNPDNRRRPWCYVQVGLKPLVQECMVHDCAD

SEQ ID NO: 6 - DNA, 258 bp, Artificial Sequence
  human derived abrogen
aaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagat

SEQ ID NO: 7 - protein, 86 aa, Artificial Sequence
  human derived abrogen
KTCYEGNGHFYRGKASTDTMGRPCLPWNSATVLQQTYHAHRSDALQLGLGKHNYCRNPDNRRRPWCYVQVGLKLLVQECMVHDCAD

SEQ ID NO: 8 - DNA, 258 bp, Artificial Sequence
  human derived abrogen
aaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagctgcttgtccaagagtgcatggtgcatgactgcgcagat

SEQ ID NO: 9 - protein, 87 aa, Artificial Sequence
  human derived fusion protein
AKTCYEGNGHFYRGKASTDTMGRPCLPWNSATVLQQTYHAHRSDALQLGLGKHNYCRNPDNRRRPWCYVQVGLKPLVQECMVHDCAD

SEQ ID NO: 10 - protein, 87 aa, Artificial Sequence
  human derived fusion protein
AKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 11 - protein, 585 aa, Artificial Sequence [sequence omitted]

SEQ ID NO: 12 - protein, 17 aa, Artificial Sequence
  human derived linker peptide
DAGGGGSGGGGSGGGGS

SEQ ID NO: 13 - protein, 689 aa, Artificial Sequence
  fusion protein human abrogen
ADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAGGGGSGGGGSGGGGSKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 14 - protein, 674 aa, Artificial Sequence
  fusion protein human abrogen
ADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 15 - protein, 672 aa, Artificial Sequence
  fusion protein human abrogen
AKTCYEGNGHFYRGKASTDTMGRPCLPWNSATVLQQTYHAHRSNALQLGLGKHNYCRNPDNRRRPWCYVQVGLKPLVQECMVHDCADDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 16 - protein, 15 aa, Artificial Sequence
  human derived linker peptide
GGGGSGGGGSGGGGS

SEQ ID NO: 17 - protein, 687 aa, Artificial Sequence
  fusion protein human abrogen
AKTCYEGNGHFYRGKASTDTMGRPCLPWNSATVLQQTYHAHRSNALQLGLGKHNYCRNPDNRRRPWCYVQVGLKPLVQECMVHDCADGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 18 - protein, 688 aa, Artificial Sequence
  fusion protein human abrogen
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAGGGGSGGGGSGGGGSKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 19 - protein, 233 aa, Artificial Sequence
  human abrogen fusion protein IgG region
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 322 aa, Artificial Sequence
  fusion protein human abrogen
ARLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 21 - protein, 322 aa, Artificial Sequence
  fusion protein human abrogen
AKTCYEGNGHFYRGKASTDTMGRPCLPWNSATVLQQTYHAHRSNALQLGLGKHNYCRNPDNRRRPWCYVQVGLKPLVQECMVHDCADRLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 86 aa, Artificial Sequence; identical to SEQ ID NO: 5
  fragment of human urokinase plasminogen activator

SEQ ID NO: 23 - DNA, 258 bp, Artificial Sequence; identical to SEQ ID NO: 6
  fragment of human urokinase plasminogen activator

SEQ ID NO: 24 - protein, 86 aa, Artificial Sequence; identical to SEQ ID NO: 3
  fragment of mouse urokinase plasminogen activator

SEQ ID NO: 25 - DNA, 258 bp, Artificial Sequence; identical to SEQ ID NO: 4
  fragment of mouse urokinase plasminogen activator cDNA

SEQ ID NO: 26 - DNA, 258 bp, Artificial Sequence
  fragment of human urokinase plasminogen activator
aaaacctgctatgaggggaatggtcacttttaccgaggaaaggccagcactgacaccatgggccggccctgcctgccctggaactctgccactgtccttcagcaaacgtaccatgcccacagatctgatgctcttcngctgggcctggggaaacataattactgcaggaacccagacaaccggaggcgaccctggtgctatgtgcaggtgggcctaaagcngcttgtccaagagtgcatggtgcatgactgcgcagat

SEQ ID NO: 27 - protein, 86 aa, Artificial Sequence
  fragment of human urokinase plasminogen activator
KTCYEGNGHFYRGKASTDTMGRPCLPWNSATVLQQTYHAHRSXALQLGLGKHNYCRNPDNRRRPWCYVQVGLKXLVQECMVHDCAD

(19) United States
(12) Patent Application Publication
(10) Pub. No.: US 2003/0228298 A1
(43) Pub. Date: Dec. 11, 2003

(54) Abrogen polypeptides, nucleic acids encoding them and methods for using them to inhibit angiogenesis

(76) Inventors: Mark Nesbit, Vincennes (FR); Timothy C. Fong, Moraga, CA (US); Dirk Brockstedt, Oakland, CA (US)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/233,675

(22) Filed: Sep. 4, 2002

Related U.S. Application Data
(60) Provisional application No. 60/316,300, filed on Sep. 4, 2001.

(51) Int. Cl. A61K 38/43; C12Q 1/68; C07H 21/04; C12N 9/99; C12P 21/02; C12N 5/06

(52) U.S. Cl. 424/94.1; 435/69.2; 435/184; 435/320.1; 435/325; 536/23.2; 435/6

(57) ABSTRACT

The invention relates to abrogen polypeptides and nucleic acids that encode them. In general, the abrogen polypeptides comprise the kringle domain from, for example, urokinase plasminogen activator. Abrogen polypeptides can be used to inhibit endothelial cell activation and/or proliferation and can inhibit endothelial cells activated or induced by both bFGF and VEGF. The invention also encompasses methods to produce polypeptides that possess abrogen activity as well as method for using these polypeptides.

### FIELD OF THE INVENTION AND INTRODUCTION

The present invention relates to novel nucleic acids encoding novel amino acid fragments of polypeptides, called abrogens. The present invention also relates to novel, potent in vitro and in vivo inhibitors of endothelial cells proliferation, and compositions of them and uses of them. The present invention further provides methods that are effective for modulating angiogenesis and inhibiting unwanted angiogenesis. Therefore, polypeptides according to the present invention are useful for treating and/or preventing cancer, tumor growth, or other angiogenic dependent or angiogenic associated diseases.

### BACKGROUND OF THE INVENTION

Angiogenesis is the generation of new blood vessels from preexisting vessels into a tissue or organ. Angiogenesis is required and normally observed under normal physiological conditions, such as for example, for wound healing, fetal and embryonic development, for female reproduction, i.e., formation of the corpus luteum, endometrium and placenta, organ formation, tissue regeneration and remodeling (Risau W et al., Nature, 1997, 386, 671-674).

Angiogenesis begins with local degradation of the basement membrane of capillaries, followed by invasion of stroma by underlying endothelial cells in the direction of an angiogenic stimulus. Subsequent to migration, endothelial cells proliferate at the leading edge of a migrating column and then organize to form new capillary tubes.

Persistent, unregulated angiogenesis occurs in a multiplicity of pathological conditions, tumor metastasis and abnormal growth by endothelial cells and supports the pathological damage seen in these conditions. The diverse pathological disease states in which unregulated angiogenesis is present have been grouped together as angiogenic dependent or angiogenic associated diseases. Outgrowth of new blood vessels under pathological conditions can lead to the development and progression of diseases such as tumor growth, diabetic retinopathy, tissue and organ malformation, obesity, macular degeneration, rheumatoid arthritis, and cardiovascular disorders.

Several studies have produced direct and indirect evidence of proof that tumor growth and metastasis are angiogenesis-dependent (Brooks et al., Cell, 1994, 79, 1154-1164; Kim K J et al., Nature, 1993, 362, 841-844). Expansion of the tumor volume requires the induction of new capillary blood vessels. Tumor cells promote angiogenesis by the secretion of angiogenic factors, in particular basic fibroblast growth factor (bFGF) (Kandel J. et al., Cell, 1991, 66, 1095-1104) and vascular endothelial growth factor (VEGF) (Ferrara et al., Endocr. Rev., 1997, 18: 4-25). Tumors may produce one or more of these angiogenic peptides that can synergistically stimulate tumor angiogenesis (Mustonen et al., J Cell Biol., 1995, 129, 865-898). Therefore, expression or administration of anti-angiogenic factors by gene therapy, for instance, should counteract the tumor-induced angiogenesis.

Various anti-angiogenic polypeptides have been discussed and used to treat human angiogenic dependent or angiogenic associated diseases. For example, angiostatin and endostatin, which are proteolytic fragments of plasminogen (Pgn) and collagen XVIII, respectively (O'Reilly et al., Cell, 1994, 79:315-328; O'Reilly et al., Cell, 1997, 88:1-20). Angiostatin contains the first four disulfide-linked structures of plasminogen, which are known as kringle structures, and which display differential effects on the suppression of the endothelial cell growth. For example, kringle 1 was shown to exhibit some inhibitory activity, while kringle 4 is an ineffective fragment. Hua L et al., (BBRC, 1999, 258 :668-673) has characterized another kringle structure within plasminogen but ouside of angiostatin, g., kringle 5. The kringle 5 was shown to inhibit endothelial cell proliferation and migration. Also, Renhai C. et al. (PNAS, 1999, Vol. 96, No. 10, pp. 5728-5733) has demonstrated a synergistic effect on endothelial inhibition when angiostatin and kringle 5 were coincubated with capillary endothelial cells. It was, however, stated that such association did not completely arrest tumor growth or tumors at a dormant stage.

The prothrombin kringle-2 domain, which is a fragment released from prothrombin by factor Xa cleavage, was identified as having anti-endothelial cell proliferative activity by Lee T H et al. (JBC, 1998, vol 273, No. 44, pp. 25505-25512) using in vitro angiogenesis assay system with bovine capillary endothelial (BCE) cell proliferation. The prothrombin kringle-2 domain was, however, described as having endothelial cell suppression activities comparable with those of angiostatin.

An amino terminal portion of the urokinase plasminogen activator uPA, termed ATF, has also been disclosed (Li et al., Hum Gen Ther 10: 3045-53, 1999; Griscelli et al., HumGenTher, 1999, Vol 10, No. 18, pp. 3045-53) as inhibiting angiogenesis. uPA is composed of three domains, a serine proteinase domain, a kringle domain, and a growth-factor-like domain. The urokinase plasminogen binds to its receptor (uPAR) by its growth-factor-like domain, and initiates a proteolytic cascade at the surface of migrating cells to stimulate intracellular signaling responsible for cell migration and proliferation. The uPA lacking the growth-factor-like domain was, however, unable to associate with uPAR and was rapidly cleared from the cell surface (Poliakov et al., Biochem J., 2001, 355:639-45).

Binding of uPA to its receptor greatly potentiates plasminogen/plasmin conversion at the cell surface. Plasmin is a broadly specific serine protease, which can directly degrade components of the extracellular matrix. uPA and plasmin are somehow involved in cell morphogenesis by activating or inducing the release of morphogenic factors, such as vascular endothelial growth factor (VEGF), hepatocyte growth factor (HGF), or fibroblast growth factor (FGF). Clinical observations correlate the presence of enhanced uPA activity at the invasive edge of the tumors (Schmitt M et al., Fibrinolysis, 1992, 6, 3-26). ATF is capable of mediating disruption of the uPA/uPAR complex and inhibiting tumor cell migration and invasion in vitro (H. Lu et al., FEBS Letter, 1994, 356, 56-59). However, the ATF molecule retains the EGF growth factor binding domain, which interacts with the uPAR receptor. Such interactions may facilitate tumor growth, as suggested in the scientific literature (Rabbani et al., J Biol. Chem 275:16450-58 (1992)).

### SUMMARY OF THE INVENTION

The present invention provides kringle-containing polypeptides, called abrogens, that are potent inhibitors of endothelial proliferation and angiogenesis. The abrogen polypeptides are capable of inhibiting or reducing cell proliferation induced by both bFGF and VEGF in a specific endothelial cell proliferation assay, whereas angiostatin only inhibits bFGF induced proliferation in this assay. Furthermore, vectors that express abrogen polypeptides in vivo reduce tumor metastasis in two lung cancer models. Thus, aspects of the invention include novel polypeptides, nucleic acids that encode them, vectors containing them, and methods of using any of these aspects to express polypeptides, alter growth or other characteristics of cells, or treat or prevent disease are provided by the invention.

Embodiments of the abrogen activity include a region of urokinase plasminogen activator encompassing the kringle domain. The mammalian urokinase plasminogen activator (uPA) kringle domain (ATF-kringle) has not been previously identified as a separate molecule with anti-angiogenic activity. Rather, it was previously shown to be a potent source of attraction of smooth muscle cells [2]. Surprisingly, we identify and show that the ATF-kringle retains a very potent anti-angiogenic activity, while not containing the growth-factor-like domain acting as binding site to the uPAR, thereby allowing uPA/uPAR complex disruption. As demonstrated in Example 3, for example, ATF-kringle containing polypeptides can inhibit endothelial cell activation and/or proliferation mediated by several different proangiogenic proteins, such as bFGF and VEGF, in a species independent manner.

The use of the kringle domain allows greater specificity in the anti-angiogenic mode of action. Our data from in vitro studies shows that the ATF-kringle molecule possesses a new activity that inhibits both bFGF and VEGF induced tube formation and/or cell proliferation in a specific endothelial cell assay. This assay also distinguishes the species-specific activity of other anti-angiogenic polypeptides. The abrogen polypeptides, and in particular those of SEQ ID No.: 1, 3, 5, and 7, do not show a species-specific response and both mouse and human derived polypeptides, for example, function in a mouse model system. This can be advantageous in developing human therapeutic compositions based upon a mouse model system. In another contrast over previous polypeptides, anti-angiogenic factors such as endostatin or angiostatin only inhibit bFGF-induced activity in this assay (Chen et al., Hum Gen Ther 11: 1983-96 (2000)). In general terms, the invention encompasses the production of, identification of, and use of polypeptides, as well as the nucleic acids that encode them, that possess this new activity, referred to as abrogens.

Thus, in one aspect, the invention comprises an isolated abrogen polypeptide, such as one with an amino acid sequence of SEQ ID NO.: 1, 3, 5, or 7 the polypeptide being in a form that does not exist in nature and has not been previously disclosed. The abrogen polypeptide can be in purified form, so that, for example, it is no longer inside a cell that produces it, it is in an extract derived from a cell that produces it, it is at least partially separated from a final reaction mixture that produces it, or one or more components of a mixture containing it have been substantially or to a measurable extent removed. A purified form can also be a form suitable for pharmaceutical research use, such as a form substantially free of antigenic or inflammatory components. A purified form can also be the result of an affinity purification process.

The invention also includes a nucleic acid comprising or consisting of a sequence that encodes an abrogen polypeptide, such as the sequences of SEQ ID NO.: 2, 4, 6, or 8. The nucleic acid can be DNA, RNA, or DNA or DNA comprising modified nucleotide bases. A nucleic acid encoding an abrogen polypeptide can also be operably linked to a variety of one or more sequences used in expression vectors, and/or cloning vectors, and/or other vectors. For example, the abrogen encoding nucleic acid can be linked to a promoter, enhancer, a sequence encoding a signal sequence, and/or a sequence encoding an affinity purification sequence. One of ordinary skill in the art is familiar with selecting appropriate sequence(s) or vector(s) and using them. The invention also encompasses cells that contain or comprise an abrogen polypeptide or abrogen encoding nucleic acid.

The cell can be transduced with, transfected with, or have an introduced into it a vector that comprises the abrogen encoding nucleic acid. Progeny of the cell, for example cells that result from cultured cell splitting or maintenance procedures, are also included in the invention. The cell can be a cultured primary cell, an established cell line cell, a transformed cell, a tumor cell, an endothelial cell, or a variety of other mammalian cells.

The invention also comprises a novel purified polypeptide that comprises a fragment of a mammalian or human kringle-containing protein, the fragment having a kringle domain that is capable of inhibiting tube formation in endothelial cell cultures induced by bFGF and VEGF, and/or capable of reducing cell proliferation induced by bFGF and VEGF, and/or capable of inhibiting metastasis of mammalian tumors. This fragment does not contain an EGF-binding domain, such as the EGF-binding domain of uPA or the amino terminal fragment (ATF) of uPA. The novel purified polypeptide does not contain the exact amino acid sequence of the kringle 5 domain of human plasminogen, the exact sequence of kringle 2 from human prothrombin, the exact 80 amino acids beginning at residue 462 of human plasminogen, or the exact sequence of any of the previously disclosed kringle-containing polypeptides, peptides, or proteins. The novel polypeptides can advantageously be used in a number of instances where inhibiting or reducing cell proliferation associated with bFGF and VEGF treatment is desired, and/or where inhibiting angiogenesis or tumor metastasis is desired.

In another aspect, the invention comprises nucleic acids that encode these novel polypeptides, vectors containing them, and cells containing them. Preferably, inhibiting tube formation in endothelial cell cultures induced by bFGF and VEGF, reducing cell proliferation induced by bFGF and VEGF, and/or inhibiting metastasis of mammalian tumors is measured in culture with established endothelial cell lines or tumor cell lines. However, other types of measurements, including measurements in vivo, can also be used. In this and other aspects of the invention involving cells, a preferred embodiment employs or involves human umbilical vein endothelial cells or mammary or lung tumor cells.

Preferably, the kringle-containing protein is human protein, such as a human plasminogen activator, like urokinase plasminogen activator or tissue plasminogen activator. Other human proteins from which the novel polypeptides and nucleic acids of the invention can be derived are ApoArgC, Factor XII, hepatocyte growth factor activator, hyaluronan binding protein, macrophage stimulating protein, thrombin, retinoic acid receptors 1 and 2, and kringle containing domains from extended sequence tag database or other database. In preferred examples, these polypeptides comprise a kringle domain having a region of SEQ ID NO.: 1 from Asn 53 to Asp 59 [NYCRNPD], and further comprises one or more regions within a particular amino acid sequence identity range to a region of SEQ ID NO.: 1, 3, 5, or 7. In particular, the regions of SEQ ID NO.: 1 that may be modified include from Cys 3 to Trp 27, Asn 53 to Cys 84, Lys 1 to Thr 2, and Ala 85 to Asp 86. However, these derivatives contain the conserved 6 Cys residues that are thought to help properly fold the kringle domain into a characteristic structure. Various regions are quite amenable to modification by substitution, deletion, and/or addition, including the region from about Asn 28 to about His 52 or Lys 51, and the terminal 2 residues from each of the N terminus and C-terminus of SEQ ID NO.: 1. Particularly preferred derivatives include those with a region of approximately 50% amino acid identity to the region of SEQ ID NO.: 1 from Cys 3 to Trp 27 and a region of approximately 40% amino acid identity to the region of SEQ ID NO.: 1 from Asn 53 to Cys 84; a region of approximately 55% amino acid identity to the region of SEQ ID NO.: 1 from Cys 3 to Trp 27 and a region of approximately 45% amino acid identity to the region of SEQ ID NO.: 1 from Asn 53 to Cys 84; a region of approximately 35% amino acid identity to the region of SEQ ID NO.: 1 from Cys 3 to Trp 27 and a region of approximately 35% amino acid identity to the region of SEQ ID NO.: 1 from Asn 53 to Cys 84. For each of the abrogen regions identified here or elsewhere in this disclosure, one of skill in the art can clearly select an optimum or desirable range or specific sequence identity difference from that listed in the previous sentence. Thus, the 50% percent amino acid identity noted here and elsewhere can also be 55%, or 60%, or 65%, or 70%, or 75%, or 80%, or 85%, or 90%, or 95%, or 98%, or from about 50-55%, or 55-60%, or 60-65%, or 65-70%, or 70-75%, or 75-80%, or 80%-85%, or 85%-90%, or 90-95%, or 95-98%, or 98-99%. Similarly, the 40% noted here or elsewhere can be 45%, 50%, and above and in various ranges as just listed, and the 35% noted here and elsewhere can be 40%, or 45% and above and in various ranges as just listed. Additional examples include an abrogen polypeptide with amino acid sequence of SEQ ID NO.: 1 modified to contain 1 to about 15 amino acid changes of substitutions, deletions, or additions, wherein the amino acid changes occur in the amino acids from Asn 28 to His 52, Lys 1 to Thr 2, Ala 85 to Asp 86. Furthermore, derivatives may merely contain or may additionally contain 1 to about 5, 1 to about 10, 1 to about 15, or 1 to about 20 amino acid changes outside of the consensus region from Asn 53 to Asp 59 of SEQ ID NO.: 1 [NYCRNPD] that are conservative amino acid substitutions.

The polypeptides and the nucleic acids that encode them may additionally have or encode a selected signal sequence region and/or an affinity purification sequence region. As used herein, the term “signal sequence or signal peptide” is understood to mean a peptide segment which directs the secretion of the abrogen polypeptides or abrogen fusion polypeptides and thereafter is cleaved following translation in the host cells. The signal sequence or signal peptide thus initiates transport of a protein across the membrane of the endoplasmic reticulum. Signal sequences have been well characterized in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. Following initiation, the signal peptide is usually cleaved within the lumen of the endoplasmic reticulum by cellular enzymes known as signal peptidases (von Heijne (1986) Nucleic Acids Res., 14: 4683). Numerous examples exist including the well known poly-His tag sequence, the immunoglobulin signal sequence, and the human interleukin 2 (IL2) signal sequence.

The polypeptide and the sequence encoding the polypeptide used in a specific vector encoding the given kringle domain may also be linked to stabilizing elements or polypeptides or the sequences that encode them, such as those from human serum albumin or the immunoglobulin Fc portion of an IgG molecule.

The abrogen polypeptides according to the present invention may be advantageously linked to a human serum albumin (HSA) or other fusion partner. Such fusion polypeptides comprise the abrogen polypeptide fused at its C- or N-terminal with HSA. The amino acid sequence of HSA is well known in the art and is inter alia disclosed by Meloun et al. (Complete Amino Acid Sequence of HSA, FEBS Letter: 58:1. 136-137, 1975) and Behrens et al. (Structure of HSA, Fed. Proc. 34,591, 1975), and more recently by genetic analysis (Lawn et al., Nucleic Acids Research, 1981, 9, 6102-6114). Shorter forms or variants of HSA, as described in EP 322 094, may also be used to produce the abrogen fusion protein of the invention. Any abrogen polypeptide noted here can be used to prepare an abrogen fusion protein or polypeptide of the invention. Construction of such fusion proteins is well known in the art and is disclosed inter alia, in U.S. Pat. No. 5,876,969. Fusion proteins so obtained possess a particularly advantageous distribution in the body, while modifying the pharmacokinetic properties of the abrogen poplypeptide and compositions containing them, and favors the development of their biological activity.

An abrogen fusion protein or polypeptide according to the present invention may also comprise an N-terminal signal peptide, such as the IL2 signal peptide providing for secretion into the surrounding medium, followed or preceded by a HSA or a portion thereof, or a variant thereof and the sequence of the abrogen polypeptides. The abrogen polypeptides may be coupled either directly or via an artificial peptide or linker to albumin, at the N-terminal end or the C-terminal end.

The chimeric molecule may be produced by eucaryotic, prokaryotic, or cellular hosts that contain a nucleotide sequence encoding the abrogen fusion protein, and then harvesting the polypeptide produced. Animal cells, yeast, fungi may be used as eucaryotic hosts. In particular, yeasts of the genus of Saccharomyces, Kluveromyces, Pichia, Schwanniomyces, or Hansenula may be cited. Animal cells, such as for example, COS, CHO, 293 cell lines, and C127 cells, and the like may be used. Fungi such as Aspergillus sp., or Trichoderma ssp may be used. Bacteria, such as Esherichia coli, or bacteria belonging to the genera of Corynebacterium, Bacillus, or Streptomyces may be used as prokaryotic cells.

In another fusion protein or polypeptide example, the abrogen polypeptide is fused to an immunoglobulin Fc region as described in WO 00/01133. Immunoglobulin Fc region is understood to mean the carboxylterminal portion of an immunoglobulin chain constant region, preferably an immunoglobulin heavy chain constant region, or a portion thereof. For example, an immunoglobulin Fc region may comprise: 1) an immunoglobulin constant heavy 1 (CH1) domain, an immunoglobulin constant heavy 2 (CH2) domain, and an immunoglobulin constant heavy (CH3) domain; 2) a CH1 domain and a CH2 domain; 3) a CH1 domain and a CH3 domain; 4) a CH2 domain and a CH3 domain; and/or 5) a combination of two or more domains and an immunoglobulin hinge region. In a preferred embodiment the Fe region used in the DNA construct encoding the abrogen polypeptide also encodes an immunoglobulin hinge region, CH2 and CH3 domains, and depending upon the type of immunoglobulin used to generate the Fc region, optionally a CH4 domain. More preferably, the immunoglobulin Fc region comprises a hinge region, and CH2 and CH3 domains. Immunoglobulin from which the heavy chain constant region is preferably derived is IgG of subclasses 1, 2, 3, or 4, and most preferably of subclass 2, most preferably the murin or human immunoglobulin Fc region from IgG2a. Other classes of immunoglobulin, IgA, IgD, IgE and IgM, may be used. The choice of appropriate or advantageous immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The Fe region used in the fusion protein is preferably from a mammalian species, for example from murine origin, and preferably from human origin, or from a humanized Fe region.

The fusion proteins of the invention preferably are generated by conventional recombinant DNA methodologies. The fusion proteins preferably are produced by expression in a host cell of a DNA molecule encoding a signal sequence, an immunoglobulin Fe region or HSA for example, and an abrogen polypeptide. The constructs may encode in a 5′ to 3′ direction, the signal sequence, the immunoglobulin Fe region or HSA for example, and the abrogen polypeptide. Alternatively, the constructs may encode in a 5′ to ₃′ direction, the signal sequence, the abrogen polypeptide and the immunoglobulin Fe region or HSA for example. As noted above, other fusion partners or stabilizing elements or polypeptides can be selected for use. The abrogen polypeptide may be coupled either directly or via a linker to the immunoglobulin Fe region or HAS, for example. The fusion of the abrogen with the immunoglobulin Fe region are produced by introducing into mammalian cell such constructs, and culturing the mammalian cells to produce the fusion proteins. The resulting fusion protein can be harvested, refolded if necessary, and purified using conventional purification techniques well known and used in the art. The resulting abrogen polypeptides exhibit longer serum half-lives, presumably due to their larger molecular sizes, and other advantageous properties.

The abrogen polypeptides and either the HSA or the immunoglobulin Fe region, for example, may be linked by a polypeptide linker. As used herein the term “polypeptide linker” is understood to mean a peptide sequence that can link two proteins together or a protein and an Fc region. The polypeptide linker preferably comprises a plurality of amino acids such as glycine and/or serine. Preferably, the polypeptide linker comprises a series of glycine and serine peptides about 10-15 residues in length. See, for example, U.S. Pat. No. 5,258,698, the disclosure of which is incorporated herein by reference. More preferably, the linker sequence is as set forth in SEQ ID NO: 12 or 16, or comprises an Asp-Ala or an Arg-Leu sequence. It is contemplated however, that the optimal linker length and amino acid composition may be determined by routine experimentation.

The present invention also provides methods for producing abrogen from non-human species as and fusion proteins, such as with HAS and Fc regions. Non-human angiogenesis inhibitor fusion proteins are useful for preclinical studies of angiogenesis inhibitors because efficacy and toxicity studies of a protein drug must be performed in animal model systems before testing in humans. A human protein may elicit an immune response in mouse, and/or exhibit different pharmacokinetics, skewing the test results. Therefore, the equivalent mouse protein is the best surrogate for the human protein for testing in a mouse model.

Additionally, various promoter/enhancer and RNA transcript stabilizing elements may be included in the vector.

In another aspect, the invention comprises methods for analyzing or identifying a polypeptide that reduces or inhibits endothelial cell proliferation induced by bFGF and VEGF, and/or reduces or inhibits tube formation induced by bFGF and VEGF, and/or reduces or inhibits tumor metastasis. In general, the method may comprise selecting a polypeptide having a kringle domain from a mammalian protein, the kringle domain comprising amino acid residues Asn 53 to Asp 59 of SEQ ID NO.: 1 [NYCRNPD], the kringle domain also containing 6 Cys residues and 2 Trp residues, and introducing the polypeptide to an endothelial cell, for example by employing an expression vector such as a recombinant adenoviral vector, a recombinant adeno-associated viral vector, or a plasmid vector. Any method for measuring the relative inhibition of tubule formation, the relative inhibition of cell proliferation, or the relative inhibition of tumor metastasis can be employed to detect a polypeptide having the appropriate characteristic or even a combination of characteristics. The invention specifically includes polypeptides and nucleic acids encoding these polypeptides that are identified or are capable of being identified by these methods.

Moreover, an abrogen polypeptide and compositions comprising it may be used as a therapeutic. The polypeptide and the method for expressing it in a cell can be, therefore, used in methods to treat or prevent a variety of angiogenesis related diseases or conditions, including, but not limited to hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, rheumatoid arthritis, diabetic neovascularization, diabetic retinopathy, macular degeneration, wound healing, obesity, peptic ulcer, Helicobacter related diseases, fractures, keloids, vasculogenesis, hematopoiesis, ovulation-related disorders, menstruation-related disorders, placentation, and cat scratch fever.

In general, the use can also be for abrogating tumor vasculature growth or angiogenesis associated with a tumor. One skilled in the art is familiar with polypeptide expression and purification systems as well as methods for administering polypeptides and vectors in appropriate pharmaceutical compositions.

In another aspect, the nucleic acids encoding an abrogen polypeptide can be used in a gene transfer method. The examples show how recombinant plasmid and adenoviral vectors, for example, can be used to affect metastasis in a lung tumor model. Various gene transfer and gene therapy vectors can be used in conjunction with the nucleic acids of the invention to either analyze the activity of an abrogen polypeptide in vivo or treat, prevent, or ameliorate an angiogenesis-related disease or condition in an animal. Preferably, the animal is human or mouse. More particularly, a nucleic acid encoding an abrogen of SEQ ID NO.: 1, 3, 5, or 7 can be cloned into a vector, preferably an adenoviral vector, an adeno-associated virus (AAV), a plasmid, or other suitable viral or non-viral vector. In one embodiment, the vector is administered to a tumor bearing or non-tumor bearing animal by direct intratumoral injection, intravenous injection, intramuscular injection, electrotransfer-mediated administration, or other suitable method. The efficacy of the abrogen expressed from the vector can be assessed in the context of, for example, reduction of the primary tumor and/or abrogation of metastatic dissemination.

Accordingly, the invention comprises gene transfer methods and methods for expressing abrogen polypeptides in a cell of an animal. These methods may comprise inserting a selected abrogen encoding sequence, such as one encoding SEQ ID NO.: 1, 3, 5, or 7, into a mammalian expression vector or the expression cassette of an appropriate vector. The vector is administered to a cell of the animal by any number of methods available, including intratumoral injection, electrotransfer, infision, subcutaneous injection, intramuscular injection, or intravenous administration. The effect of the expressed abrogen polypeptide can then be measured and compared to control. These methods can be used to treat any one of a number of angiogenesis related diseases or disorders, such as those listed above.

The invention also comprises administration of the abrogen recombinant polypeptides in a cell of an animal. These methods may comprise administering the abrogen peptide as in SEQ ID NO: 1, 3, 5, 7 by any well-known method in the art, including for example, direct injections of the peptide at a specific site, i.e., by ophthalmic (including intravitreal or intraorbital), intraperitoneal, intramuscular, or intratumoral injections.

The invention also includes compositions comprising the abrogen polypeptides or nucleic acids, and the derivatives and nucleic acids encoding derivatives, such as those having the sequences of SEQ ID NO.: 1-8 or SEQ ID NO.: 9, 10, 13, 14, 15, 17, 18, 20, or 21, or abrogen fusion polypeptides or nucleic acids encosing them. The abrogen polypeptides or derivatives can be recombinant polypeptides or purified polypeptides. The compositions of the present invention may be provided to an animal by any suitable means, directly (ea., locally, as by injection, implantation or topical administration to a tissue locus) or systemically (e.g., parenterally or orally). Where the composition is to be provided parenterally, such as by intravenous, subcutaneous, ophthalmic (including intravitreal or intracameral), intraperitoneal, intramuscular, buccal, rectal, vaginal, intraorbital, intracerebral, intracranial, intraspinal, intraventricular, intrathecal, intracistemal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline (e. g., 9.85% aqueous NaCl, 0.15 M, pH 7-7.4). In one embodiment, the composition is a pharmaceutically acceptable composition. One skilled in the art is familiar with selecting and testing pharmaceutically acceptable compositions for use with recombinant polypeptides and nucleic acids.

The abrogen formulations may conveniently be presented in unit dosage form and may be prepared by conventional pharmaceutical techniques. Such techniques include the step of bringing into association the active ingredient and the pharmaceutical carrier(s) or excipient(s).

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

It is another aspect or object of the present invention to provide a method of treating diseases and processes that are mediated by angiogenesis.

It is yet another aspect of the present invention to provide a method and composition for treating diseases and processes that are mediated by angiogenesis including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, rheumatoid arthritis, diabetic neovascularization, diabetic retinopathy, macular degeneration, wound healing, peptic ulcer, Helicobacter related diseases, fractures, keloids, vasculogenesis, hematopoiesis, ovulation, menstruation, placentation, obesity and cat scratch fever.

It is another aspect of the present invention to provide a composition for treating or repressing the growth of a cancer.

It is still another aspect of the present invention to provide a method for treating ocular angiogenesis related diseases such as macular degeneration or diabetic retinopathy by direct ophthalmic injections of the recombinant abrogen peptides.

Another aspect of the present invention is to provide a method for targeted delivery of abrogen compositions to specific locations.

Yet another aspect of the invention is to provide compositions and methods useful for gene therapy for the modulation of angiogenic processes.

Throughout this disclosure, applicants refer to journal articles, patent documents, published references, web pages, sequence information available in databases, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples that follow are merely exemplary of the scope of this invention and content of this disclosure. One skilled in the art can devise and construct numerous modifications to the examples listed below without departing from the scope of this invention.

### BRIEF DESCRIPTION OF THE FIGURES

FIG. 1: The proliferative response of transduced HUVEC human endothelial cells to human abrogen (hATF-K; SEQ ID NO. 1) and mouse abrogen (mATK-K; SEQ ID NO.: 3). Cultured cells were transduced with adenoviral vectors containing an expression cassette for producing the abrogen polypeptide (hATF-K and mATF-K), a control, CMV promoter only vector (CMV), and the full amino terminal fragment of plasminogen (hATF or mATF). In FIG. 1A, the left axis indicates the degree of cell proliferation and each of the boxes represents the level of cell proliferation under a treatment regimen as indicated by the addition of bFGF, VEGF, or both. The reduction in cell proliferation in all samples where the human abrogen polypeptide is expressed (hATF-K) is markedly reduced compared to controls (CMV, HATF, and mATF). The proliferation in the mouse abrogen expressing cells (mATF-K) is also markedly reduced. Figure lB shows representative cell cultures from mouse and human full ATF polypeptides and mouse and human ATF-Kringle containing abrogen polypeptides (see Examples). The first page shows Control (full human ATF treated with FGF) compared to hATF-Kringle containing polypeptide treated with FGF. The remaining pages list the adenoviral vector used to transduce the cells (see Examples).

FIG. 2: Various human protein sequences having a kringle domain possessing the consensus region from Asn 53 to Asp 59 of SEQ ID NO.: 1 and the with the 6 conserved Cys, 2 conserved Trp, and conserved Gly and Arg residues aligned. These proteins and homologs, isoforms, and derivatives of them, can be used in methods of the invention.

FIG. 3: Effect of anti-angiogenic polypeptides on tubule growth in endothelial cells. Because culture conditions rapidly deplete anti-angiogenic factors if they are added as a recombinant or purified polypeptide, HUVECs are directly transduced with adenoviral vectors to provide consistent protein expression and secretion for the duration of the assay (7-10 days). HUVECs are transduced with Adenovirus expressing: human abrogen, hATF-K (as in SEQ ID NO.: 1), mouse abrogen, mATF-K (as in SEQ ID NO.: 3), and human endostatin (FIG. 3A) or human Angiostatin (FIG. 3B). Control adenovirus containing the LacZ or no gene of interest (empty control) is also included. The transduced cells are then cultured in a 3-dimensional matrix of fibrin with recombinant VEGF or bFGF added, as indicated. Tubule formation as a marker for activation and proliferation of endothelial cells is then visualized and recorded. Tubule formation in both the bFGF and VEGF treated cells is markedly inhibited in only the abrogen expressing cultures.

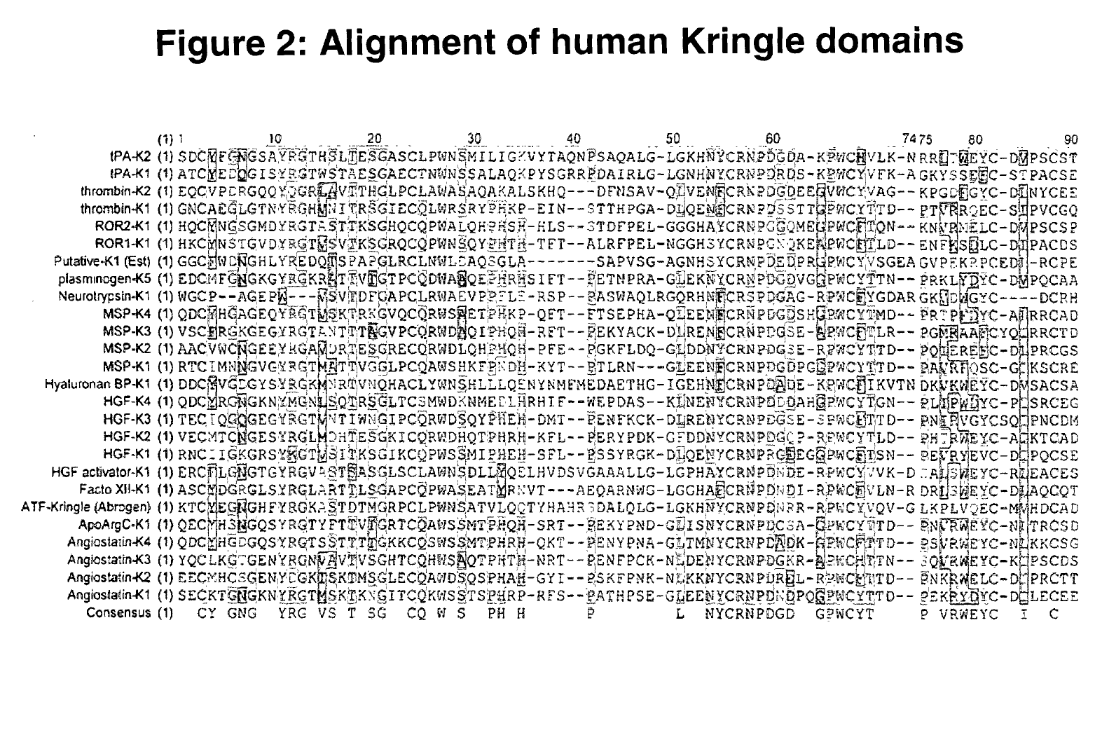

FIG. 4: Prevention of tumor metastasis in mouse 4T1 lung cancer model. Control empty plasmid and abrogen (hATF-K or mATF-K) expression cassette containing plasmid introduced via electrotransfer 6 days prior to injection of 4T1 tumor cells. Approximately 250,000 tumor cells are injected subcutaneously. Fifteen days after injection, primary tumors are removed in a surgical procedure. Lungs are harvested 35 days post tumor injection and the size and number of metastatic tumor colonies measured.

FIG. 5: Prevention of tumor metastasis in mouse 4T1 lung cancer model. Control empty plasmid compared to mATF-K expression plasmid. The assay protocol is the same as in FIG. 4.

FIG. 6: Prevention of tumor metastasis in mouse 3LL Boston lung cancer model. Control empty plasmid compared to mATF-K expression plasmid. The assay protocol is the same as FIG. 4, with the exception that 3LL Boston cells are used.

FIG. 7: Prevention of tumor metastasis in mouse 3LL Boston lung cancer model. Control empty plasmid compared to experimental control mEndostatin expression plasmid. The assay protocol is the same as FIG. 6.

FIG. 8: Measurement of size and number of metastasis in the 4T1 lung tumor model described for FIG. 4. Each spot represents the weight of the lung from each animal surveyed (C57BL/6 mice), indicating the relative size of the tumor nodules present. The left axis indicates the number of visible tumor nodules for each of the animals. With the exception of one animal in the HATF-K sample, the abrogen expressing vector treatment animals show a reduction in both the size and number of metastatic tumor nodules as compared to control. The hATF-K animals with abnormally high number of nodules were not further examined for experimental or procedural error or expression of hATF-K. Here the controls are empty plasmid (Control) and an alkaline phosphatase expressing control plasmid (mSEAP).

FIG. 9: Measurement of size and number of metastasis in the 3LL Boston lung tumor model described for FIG. 4 using the graphical representation method described for FIG. 7. Controls are the same as in FIG. 7. Again, the use of both the mouse and human abrogen expressing vectors (mATF-K and HATF-K) results in significant reduction in tumor metastasis.

FIG. 10: Measurement of size and number of metastasis in the 3LL Boston lung tumor model as described for FIG. 9. These data indicate that treatment with mouse endostatin or angiostatin, or either mouse or human ATF-K, reduce the number and size of the lung metastatic nodules compared to control treatment. The fact that both mouse and human abrogen encoding vectors are efficacious indicates that the species-specific characteristics that limit the use of the endostatin and angiostatin polypeptides are not present in the abrogen polypeptides. Furthermore, the abrogen polypeptides appear at least as efficacious as the either endostatin or angiostatin and much more efficacious than a combined endostatin/angiostatin treatment (mEndo/mAngio).

FIG. 11: Systemic expression of mouse or human derived abrogen polypeptides (here listed as MuPAK or HuPAK) from vector introduced into muscle significantly reduces the formation of spontaneous lung metastases in the 3LL-B tumor model. Systemic expression of therapeutic transgenes from the muscle is established 6 days before C57BL/6 mice are injected with a tumorigenic dose of 3LL-B tumor cells. The primary tumor is carefully excised 15 days post cell injection. The study is terminated on day 35 and lung metastases were counted. Panel A: lungs from mice treated with empty expression vector; Panel B: mice treated with human derived ATF-Kringle abrogen expressing vector (HuPAK); and Panel C: with treated with mouse derived ATF-Kringle abrogen expressing vector (MuPAK); Panel D: graphically shows the number and size of metastatic nodules present as the diameter of each “bubble” represents the lung weight.

FIG. 12: Systemic expression of mouse or human abrogen (here listed as MuPAK or HuPAK) from muscle significantly reduces the formation of spontaneous lung metastases in the MDA-MB-435 tumor model. Systemic expression of therapeutic transgenes from the muscle is established 10 days after SCID/bg mice are injected with a tumorigenic dose of MDA-MB-435 (human breast adenocarcinoma tumor cells). The primary tumor is carefully excised when a volume of 250 to 350 mm3 is reached. The study is terminated on day 89 and lung metastases measured. Panel A: lungs from mice treated with control mSEAP; Panel B: with treated with mouse derived ATF-Kringle abrogen expressing vector (here MUPAK); Panel C: mice treated with human derived ATF-Kringle abrogen expressing vector (HuPAK); and Panel D: graphically shows lung metastases counts as noted above.

FIG. 13A: is a schematic representation of the plasmid pXL2996.

FIG. 13B: is a schematic representation of the plasmid pMB063.

FIG. 13C is a schematic representation of the plasmid pBA140.

FIG. 14: is a schematic representation of the plasmid pMB060 and fusion construct.

FIG. 15: is a schematic representation of the plasmid pMB059 and fusion construct.

FIG. 16 is a schematic representation of the plasmid pMB056 and fusion construct.

FIG. 17: is a schematic representation of the plasmid pMB055 and fusion construct.

FIG. 18: is a schematic representation of the plasmid pMB060m prepro and fusion construct.

FIG. 19: is a schematic representation of the plasmid pMB053 and fusion construct.

FIG. 20: is a schematic representation of the plasmid pMB057 and fusion construct.

FIG. 21: is a schematic representation of the plasmid pXL4128.

### DETAILED DESCRIPTION OF EXEMPLARY EMBODIMENTS

A number of Kringle domain containing proteins and polypeptides have been described and used in a variety of methods, including therapeutic methods. As shown here, a Kringle-containing abrogen polypeptide can be identified and used to inhibit or reduce tumor metastasis, inhibit or reduce endothelial cell proliferation, and/or inhibit or reduce endothelial cell tubule formation. As an abrogen polypeptide or nucleic acid encoding an abrogen polypeptide, specific examples include the mouse or human derived kringle domains of uPA (SEQ ID NO.: 1-8). Additional examples have been mentioned and/or are described below in their structure and/or method of making and identifying. Functionally, an abrogen polypeptide can be distinguished by the ability to inhibit tumor metastasis. A more specific set of abrogen polypeptides include those that inhibit the endothelial cell proliferation induced by both of bFGF and VEGF, either in separate assays or together in one assay. An abrogen polypeptide can be either secreted or expressed inside a cell.

In making and using aspects and embodiments of this invention, one skilled in the art may employ conventional molecular biology, cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, N.Y. and Sambrook et al., Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription And Translation, Hames & Higgins, eds. (1984); Animal Cell Culture (R I. Freshney, ed. (1986)); Immobilized Cells And Enzymes (IRL Press, (1986)); Gennaro et al. (eds.) Remington's Pharmaceutical Sciences, 18th edition; B. Perbal, A Practical Guide To Molecular Cloning (1984); F. M. Ausubel et al. (eds.), Current Protocols in Molecular Biology, John Wiley & Sons, Inc. (2001), Coligan et al. (eds.), Current Protocols in Immunology, John Wiley & Sons, Inc. (2001); W. Paul et al. (eds.) Fundamental Immunology, Raven Press; E. J. Murray et al. (ed.) Methods in Molecular Biology: Gene Transfer and Expression Protocols, The Humana Press Inc. (1991); J. E. Celis et al., Cell Biology: A Laboratory Handbook, Academic Press (1994); J. E. Coligan et al. (Eds.) Current Protocols in Protein Science, John Wiley & Sons (2001); and J. S. Bonifacino et al. (Eds.) Current Protocols in Cell Biology, John Wiley & Sons, Inc. (2001). Additional information sources are listed below or are referred to by citation number corresponding to the references at the end of the specification.

As used herein, a “vector” means any nucleic acid or nucleic acid-bearing particle, cell, or organism capable of being used to transfer a nucleic acid into a host cell and/or used to cause the expression of a polypeptide in a host cell. The term “vector” includes both viral and nonviral products and means for introducing the nucleic acid into a cell. A “vector” can be used in vitro, ex vivo, or in vivo. Non-viral vectors include plasmids, cosmids, and can comprise liposomes, electrically charged lipids (cytofectins), DNA protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein

The abrogen derivatives of this invention include those having one or more conservative amino acid substitutions. For example, one or more amino acid residues within a sequence can be substituted by another amino acid of a similar polarity, which acts as a functional equivalent when the substitution results in no significant change in activity in at least one selected biological activity or function.

Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

“Isolated,” when referring to a nucleic acid or polypeptide, means that the indicated molecule is present in the substantial absence of at least one other molecule with which it naturally occurs or necessarily occurs because of its method of preparation. Thus, for example, an “isolated abrogen polypeptide” refers to a molecule substantially free of a macromolecule existing in a cell used to produce the abrogen polypeptide. However, the preparation or sample containing the molecule may include other components of different types. In addition, “isolated from” a particular molecule may also mean that a particular molecule is substantially absent from a preparation or sample. Varying degrees of isolation can be prepared from methods known in the art. Similarly, a “purified” form of a molecule is at least partially separated from a final reaction mixture that produces it, or one or more components of a mixture containing it have been substantially or to a measurable extent removed. A purified form can also be a form suitable for pharmaceutical research use, such as a form substantially free of antigenic or inflammatory components. A purified form can also be the result of an affinity purification process or any other purification step or process.

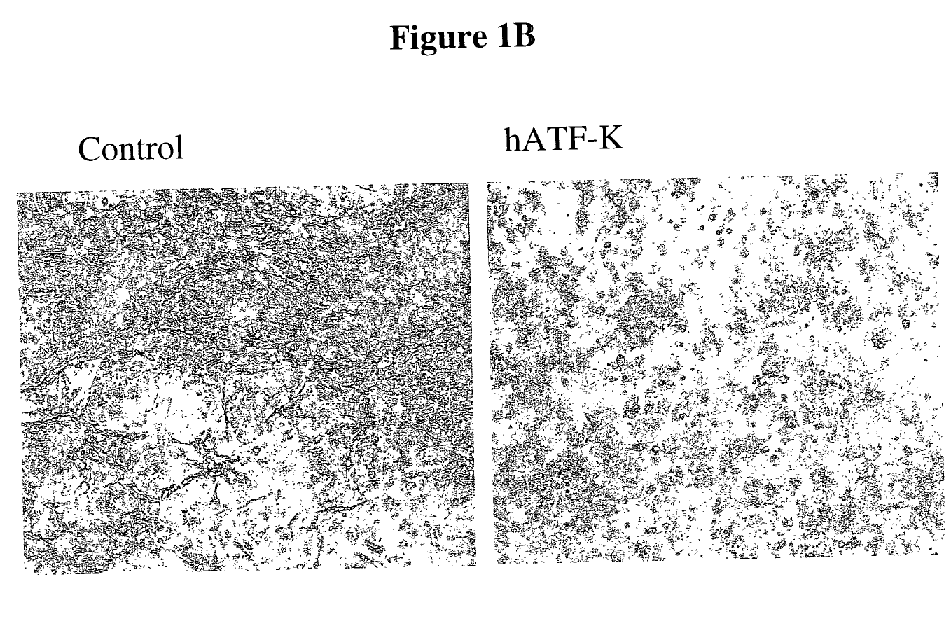

The “derivatives” noted here can be produced using homolog sequences, modifications of an existing sequence, or a combination of the two. The term “homolog” is used herein to refer to similar or homologous sequences, whether or not any particular position or residue is identical to or different from the molecule similarity or homology is measured against. A nucleic acid or amino acid sequence alignment may include spaces. Preferably, alignment is made using the consensus residues listed in FIG. 2, or the 6 Cys residues of the kringle domain. One way of defining a homolog is through “percent identity” between two nucleic acids or two polypeptide molecules. This refers to the percent defined by a comparison using a basic blastn or blastp or blastx algorithm at the default setting, unless otherwise indicated (see, for example, NCBI BLAST home page: http://www.ncbi.nlm.nih.gov/BLAST/). Aligning a Cys residue in abrogen, for example, can be performed by comparing sequences where the first amino acid residue or codon is for a particular Cys, or where the particular Cys residue is set at the same position as that of the abrogen Cys residue. For example, the blastp algorithm was used to generate homolog sequences, as in those of FIG. 2, by selecting the Blosum62 matrix, gap costs set at Existence: 11 and Extension: 1 (the default settings when performed). Typically, the default setting is used unless otherwise indicated. “Homology” can be determined by a direct comparison of the sequence information between two polypeptide molecules by aligning the sequence information and using readily available computer programs. Alternatively, homology can be determined by hybridization of polynucleotides under conditions allowing for the formation of stable duplexes between homologous regions and determining of identifying double-stranded nucleic acid.

A “functional homolog” or a “functional equivalent” of a given polypeptide or sequence includes molecules derived from the native polypeptide sequence, as well as recombinantly produced or chemically synthesized polypeptides, which function in a manner similar to the reference molecule or achieve a similar desired result. Thus, a “functional homolog” or a “functional equivalent” of a given kringle nucleotide region includes similar regions derived from a different species, nucleotide regions derived from an isoform, or from a different cellular source, or resulting from an alternative splicing event, as well as recombinantly produced or chemically synthesized nucleic acids that function in a manner similar to the reference nucleic acid region in achieving a desired result, such as a result in a particular assay or cell characteristic.

A “recombinant” molecule is one that has undergone at least one molecular biological manipulation, as known in the art. Typically, this manipulation occurs in vitro but it can also occur within a cell, as with homologous recombination. A recombinant polypeptide is one that is produced from a recombinant DNA or nucleic acid. A “coding sequence” or “sequence that encodes” is a sequence capable of being transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus.

A “nucleic acid” is a polymeric compound comprised of covalently linked nucleotides, from whatever source. Nucleic acid includes polyribonucleic acid (RNA) and polydeoxyribonucleic acid (DNA), both of which may be single-stranded or doublestranded. DNA includes cDNA, genomic DNA, synthetic DNA, and seri-synthetic DNA. The term “nucleic acid” also captures sequences that include any of the known base analogues of DNA and RNA.

A cell has been “transfected” by a vector or exogenous or heterologous nucleic acid when the vector or nucleic acid has been introduced inside the cell. A cell has been “transformed” or “transduced” by a vector or exogenous or heterologous nucleic acid when the vector or nucleic acid effects a phenotypic change or detectable modification in the cell, such as expression of a polypeptide.

As noted, the kringle-containing fragments can be selected from or derived from any available kringle-containing protein or polypeptide. Angiostatin contains kringle domains 1-4 of plasminogen and a separate kringle 5 domain exists. In studies by various investigators, individual kringle domains 1, 2 and 3 are found to have some anti-angiogenic activity and to abrogate the growth of tumors in mice [12], and the same was found for the individual kringle 5 domain [13]. Kringle homology domains are currently found in 156 different proteins. Kringle domains in plasminogen, thrombin, and hepatocyte growth factor have been shown to be anti-angiogenic [14]. The kringle-2 domain of prothrombin was recently shown to have growth inhibitory activity towards basic fibroblast growth factor stimulated capillary endothelial cells [15, 16]. It has also been shown that a kringle domain of hepatocyte growth factor is also anti-angiogenic and abrogated endothelial cell growth [17]. The abrogen polypeptides and derivatives of the invention do not have the exact same amino acid sequence of any of these previously discussed polypeptides. However, one of skill in the art may use the sequence information and functionally activity information available from these studies to construct abrogen polypeptides and derivatives, as known in the art.

Another family member is that of uPA, from which the ATF polypeptide noted above is derived. The ATF molecule still contains the EGF like growth factor domain at the N-terminus of the molecule, followed by a kringle domain. ApoArgC, Factor XII, Hepatocyte growth factor activator, hyaluronan binding protein, macrophage stimulating protein (kringles 1-4), thrombin (kringles 1 and 2), tissue type plasminogen activator (tPA) (kringles 1 and 2), retinoic acid receptors 1 and 2, and kringle domains from a protein defined in an expressed sequence tag database can all be selected for use. FIG. 2 lists an additional source of human kringle domains. A consensus kringle domain containing fragment is listed at the bottom of FIG. 2 and this consensus sequence can be used to construct derivatives of the specific abrogen polypeptides disclosed here. One skilled in the art is familiar with methods of identifying further homologs or isoforms to select and use.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 7:980-990 (1992)). Preferably, the viral vectors are replication defective or conditionally replication defective, that is, they are unable to replicate autonomously in the target cell or unable to replicate autonomously under certain conditions. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome necessary for encapsulating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 2:320-330 (1991)), defective herpes virus vector lacking a glyco-protein L gene, or other defective herpes virus vectors (PCT Publication WO 94/21807 and WO 92/05263); an attenuated adenovirus vector, such as the vector described by Stratford-Perricaudet et al. (J. Clin. Invest. 90:626-630 (1992); see also La Salle et al., Science 259:988-990 (1993)); a defective adeno-associated virus vector (Samulski et al., J. Virol. 61:3096-3101 (1987); Samulski et al., J. Virol. 63:3822-3828 (1989); Lebkowski et al., Mol. Cell. Biol. 8:3988-3996 (1988)); and a conditional replicative recombinant vectors (see, for example, U.S. Pat. Nos. 6,111,243, 5,972,706, and published PCT documents WO 00136650, WO 0024408).

Recombinant adenoviruses display many advantages for use as transgene expression systems, including a tropism for both dividing and non-dividing cells, minimal pathogenic potential, ability to replicate to high titer for preparation of vector stocks, and the potential to carry large inserts (see e.g., Berkner, K. L., Curr. Top. Micro. Immunol., 158:39-66 (1992); Jolly D., Cancer Gene Therapy, 1:51-64 (1994)).

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter or eletrotransfer device (see, e.g., Wu et al., J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990; Williams et al., Proc. Natl. Acad. Sci. USA 88:2726-2730 (1991)). Receptor-mediated DNA delivery approaches can also be used (Curiel et al., Hum. Gene Ther. 3:147-154 (1992); Wu and Wu, J. Biol. Chem. 262:4429-4432 (1987)). Naked plasmids or cosmids can be used in a number of gene transfer protocols and these plasmids and cosmids can be used in embodiments of this invention (see, in general, Miyake et al., PNAS 93:1320-1324 (1996); U.S. Pat. No. 6,143,530; U.S. Pat. No. 6,153,597; Ding et al., Cancer Res., 61:526-31 (2001); and Crouzet et al., PNAS 94:1414-1419 (1997). Among the preferred plamid vectors are those described in WO9710343 and WO9626270. Plasmids can also be combined with lipid compositions, pharmaceutically acceptable vehicles, and used with electrotransfer technology, as known in the art (see, for example, U.S. Pat. Nos. 6,156,338 and 6,143,729, and WO9901157 and the related devices in WO9901175).

### EXAMPLES

Previous studies have shown that the ATF molecule can be effective as an anti-tumoral and anti-angiogenic molecule especially when delivered by gene therapy vectors [6]. However the presence of the EGF like domain may lead to the activation of intracellular pathways in both tumor cells [3] and endothelial cells [7]. These activities are counterproductive to an anti-angiogenic treatment. We have assessed the potency of the kringle domain from human and mouse uPA (ATF-kringle) by in vitro and in vivo assays for its potential as an anti-angiogenic therapeutic. The kringle domain of human uPA was previously shown to be a potent source of attraction for smooth muscle cells [2]. This activity again is counterproductive to use as an anti-angiogenic agent. Surprisingly, our data now shows that ATF-kringle containing polypeptides can inhibit endothelial cell activation and/or proliferation mediated by several different proangiogenic proteins, such as basic fibroblast growth factor (bFGF) and vascular endothelial growth factor (VEGF), and in a species independent manner. We have designated the name Abrogen to this activity.

### Example 1

Cloning and Manipulating Abrogen Nucleic Acids.

Exemplary primary nucleotide and polypeptide structures for both the mouse and human abrogens sequences are shown below.

| | |
|---|---|
| ktc yeg ngh fyr gka std tmg rpc lpw nsa tvl qqt yha hrs nal qlg | SEQ ID NO.:1 |
| lgk hny crn pdn rrr pwc yvq vgl kpl vqe cmv hdc ad | |
| aaaacctgct atgaggggaa tggtcacttt taccgaggaa aggccagcac tgacaccatg | SEQ ID NO.:2 |
| ggccggccct gcctgccctg gaactctgcc actgtccttc agcaaacgta ccatgcccac | |
| agatctaatg ctcttcagct gggcctgggg aaacataatt actgcaggaa cccagacaac | |
| cggaggcgac cctggtgcta tgtgcaggtg ggcctaaagc cgcttgtcca agagtgcatg | |
| gtgcatgact gcgcagat | |
| ktc yhg ngd syr gka ntd tkg rpc law nap avl qkp yna hrp dai slg | SEQ ID NO.:3 |
| lgk kny crn pdn qkr pwc yvq igl rqf vqe cmv hdc sl | |
| aaaacctgct atcatggaaa tggtgactct taccgaggaa aggccaacac tgataccaaa | SEQ ID NO:4 |
| ggtcggccct gcctggcctg gaatgcgcct gctgtccttc agaaacccta caatgcccac | |
| agacctgatg ctattagcct aggcctgggg aaacacaatt actgcaggaa ccctgacaac | |
| cagaagcgac cctggtgcta tgtgcagatt ggcctaaggc agtttgtcca agaatgcatg | |
| gtgcatgact gctctctt | |
| ktc yeg ngh fyr gka std tmg rpc lpw nsa tvl qqt yha hrs dal qlg | SEQ ID NO.:5 |
| lgk hny crn pdn rrr pwc yvq vgl kpl vqe cmv hdc ad | |
| aaaacctgct atgaggggaa tggtcacttt taccgaggaa aggccagcac tgacaccatg | SEQ ID NO:6 |
| ggccggccct gcctgccctg gaactctgcc actgtccttc agcaaacgta ccatgcccac | |
| agatctgatg ctcttcagct gggcctgggg aaacataatt actgcaggaa cccagacaac | |
| cggaggcgac cctggtgcta tgtgcaggtg ggcctaaagc cgcttgtcca agagtgcatg | |
| gtgcatgact gcgcagat | |
| ktc yeg ngh fyr gka std tmg rpc lpw nsa tvl qqt yha hrs dal qlg lgk | SEQ ID NO:7 |
| hny crn pdn rrr pwc yvq vgl kll vqe cmv hdc ad | |
| aaaacctgct atgaggggaa tggtcacttt taccgaggaa aggccagcac tgacaccatg | SEQ ID NO:8 |
| ggccggccct gcctgccctg gaactctgcc actgtccttc agcaaacgta ccatgcccac | |
| agatctgatg ctcttcagct gggcctgggg aaacataatt actgcaggaa cccagacaac | |
| cggaggcgac cctggtgcta tgtgcaggtg ggcctaaagc tgcttgtcca agagtgcatg | |
| gtgcatgact gcgcagat | |

Exemplary polypeptide sequences of the fusion proteins comprising the human abrogen having sequence of SEQ ID NO: 1 fused to the IL-2 signal peptide and to human serum albumin or immunoglobulin IgG2 Fe region, as well as linker peptide sequences, are listed below.

| | |
|---|---|
| AKTCYEGNGH FYRGKASTDT MCRPCLPWNS ATVLQQTYHA HRSDALQLGL | SEQ ID NO:9 |
| GKHNYCRNPD NRRRPWCYVQ VGLKPLVQEC MVHDCAD | |
| AKTCYEGNGH FYRGKASTDT MGRPCLPWNS ATVLQQTYHA HRSNALQLGL | SEQ ID NO:10 |
| GKHNYCRNPD NRRRPWCYVQ VGLKPLVQEC MVHDCAD | |
| DAHKSEVAH RFKDLGEENF KALVLIAFAQ YLQQCPFEDH VKLVNEVTEF | SEQ ID NO:11 |
| AKTCVADESA ENCDKSLHTL FGDKLCTVAT LRETYGEMAD CCAKQEPERN | |
| ECFLQHKDDN PNLPRLVRPE VDVMCTAFHD NEETFLKKYL YEIARRHPYF | |
| YAPELLFFAK RYKAAFTECC QAADKAACLL PKLDELRDEC KASSAKQRLK | |
| CASLQKFGER AFKAWAVARL SQRFPKAEFA EVSKLVTDLT KVHTECCHGD | |
| LLECADDRAD LAKYICENQD SISSKLKECC EKPLLEKSHC IAEVENDEMP | |
| ADLPSLAADF VESKDVCKNY AEAKDVFLGM FLYEYARRHP DYSVVLLLRL | |
| AKTYETTLEK CCAAADPHEC YAKVFDEFKP LVEEPQNLIK QNCELFEQLG | |
| EYKFQNALLV RYTKKVPQVS TPTLVEVSRN LGKVGSKCCK HPEAKRMPCA | |
| EDYLSVVLNQ LCVLHEKTPV SDRVTKCCTE SLVNRRPCFS ALEVDETYVP | |
| KEFNAETFTF HADICTLSEK ERQIKKQTAL VELVKHKPKA TKEQLKAVMD | |
| DFAAFVEKCC KADDKETCFA EEGKKLVAAS QAALGL | |
| DAGGGGSGGGGSGGGGS | SEQ ID NO:12 |
| ADAHKSEVAH RFKDLGEENF KALVLIAFAQ YLQQCPFEDH VKLVNEVTEF | SEQ ID NO:13 |
| AKTCVADESA ENCDKSLHTL FGDKLCTVAT LRETYGEMAD CCAKQEPERN | |
| ECFLQHKDDN PNLPRLVRPE VDVMCTAFHD NEETFLKKYL YEIARRHPYF | |
| YAPELLFFAK RYKAAFTECC QAADKAACLL PKLDELRDEG KASSAKQRLK | |
| CASLQKFGER AFKAWAVARL SQRFPKAEFA EVSKLVTDLT KVHTECCHGD | |
| LLECADDRAD LAKYICENQD SISSKLKECC EKPLLEKSHC IAEVENDEMP | |
| ADLPSLAADF VESKDVCKNY AEAKDVFLGM FLYEYARRHP DYSVVLLLRL | |
| AKTYETTLEK CCAAADPHEC YAKVFDEFKP LVEEPQNLIK QNCELFEQLG | |
| EYKFQNALLV RYTKKVPQVS TPTLVEVSRN LGKVGSKCCK HPEAKRMPCA | |
| EDYLSVVLNQ LCVLHEKTPV SDRVTKCCTE SLVNRRPCFS ALEVDETYVP | |
| KEFNAETFTF HADICTLSEK ERQIKKQTAL VELVKHKPKA TKEQLKAVMD | |
| DFAAFVEKCC KADDKETCFA EEGKKLVAAS QAALGLDAGG GGSGGGGSGG | |
| GGSKTCYEGN GHFYRGKAST DTMGRPCLPW NSATVLQQTY HAHRSNALQL | |
| GLGKHNYCRN PDNRRRPWCY VQVGLKPLVQ ECMVHDCAD | |
| ADAHKSEVAH RFKDLGEENF KALVLIAFAQ YLQQCPFEDH VKLVNEVTEF | SEQ ID NO:14 |
| AKTCVADESA ENCDKSLHTL FGDKLCTVAT LRETYGEMAD CCAKQEPERN | |
| ECFLQHKDDN PNLPRLVRPE VDVMCTAFHD NEETFLKKYL YEIARRHPYF | |
| YAPELLFFAK RYKAAFTECC QAADKAACLL PKLDELRDEG KASSAKQRLK | |
| CASLQKFGER AFKAWAVARL SQRFPKAEFA EVSKLVTDLT KVHTECCHGD | |
| LLECADDRAD LAKYICENQD SISSKLKECC EKPLLEKSHC IAEVENDEMP | |
| ADLPSLAADF VESKDVCKNY AEAKDVFLGM FLYEYARRHP DYSVVLLLRL | |
| AKTYETTLEK CCAAADPHEC YAKVFDEFKP LVEEPQNLIK QNCELFEQLG | |
| EYKFQNALLV RYTKKVPQVS TPTLVEVSRN LGKVGSKCCK HPEAKRMPCA | |
| EDYLSVVLNQ LCVLHEKTPV SDRVTKCCTE SLVNRRPCFS ALEVDETYVP | |
| KEFNAETFTF HADICTLSEK ERQIKKQTAL VELVKHKPKA TKEQLKAVMD | |
| DFAAFVEKCC KADDKETCFA EEGKKLVAAS QAALGLDAKT CYEGNGHFYR | |
| GKASTDTMGR PCLPWNSATV LQQTYHAHRS NALQLGLGKH NYCRNPDNRR | |
| RPWCYVQVGL KPLVQECMVH DCAD | |
| AKTCYEGNGH FYRGKASTDT MGRPCLPWNS ATVLQQTYHA HRSNALQLGL | SEQ ID NO:15 |
| GKHNYCRNPD NRRRPWCYVQ VGLKPLVQEC MVHDCADDAH KSEVAHRFKD | |
| LGEENFKALV LIAFAQYLQQ CPFEDHVKLV NEVTEFAKTC VADESAENCD | |
| KSLHTLFGDK LCTVATLRET YGEMADCCAK QEPERNECEL QHKDDNPNLP | |
| RLVRPEVDVM CTAFHDNEET FLKKYLYEIA RRHPYFYAPE LLFFAKRYKA | |
| AFTECCQAAD KAACLLPKLD ELRDEGKASS AKQRLKCASL QKFGEPAFKA | |
| WAVARLSQRF PKAEFAEVSK LVTDLTKVHT ECCHGDLLEC ADDRADLAKY | |
| ICENQDSISS KLKECCEKPL LEKSHCIAEV ENDEMPADLP SLAADFVESK | |
| DVCKNYAEAK DVFLGMFLYE YARRHPDYSV VLLLRLAKTY ETTLEKCCAA | |
| ADPHECYAKV FDEFKPLVEE PQNLIKQNCE LFEQLGEYKF QNALLVRYTK | |
| KVPQVSTPTL VEVSRNLGKV GSKCCKHPEA KRMPCAEDYL SVVLNQLCVL | |
| HEKTPVSDRV TKCCTESLVN RRPCFSALEV DETYVPKEFN AETETEHADI | |
| CTLSEKERQI KKQTALVELV KHKPKATKEQ LKAVMDDFAA FVEKCCKADD | |
| KETCFAEEGK KLVAASQAAL CL | |
| GGGGSGGGGSGGGGS | SEQ ID NO:16 |
| AKTCYEGNGH FYRGKASTDT MGRPCLPWNS ATVLQQTYHA HRSNALQLGL | SEQ ID NO:17 |
| GKHNYCRNPD NRRRPWCYVQ VCLKPLVQEC MVHDCADGGG GSGCGGSGGG | |
| CSDAHKSEVA HRFKDLGEEN FKALVLIAFA QYLQQCPFED HVKLVNEVTE | |
| FAKTCVADES AENCDKSLHT LFGDKLCTVA TLRETYGEMA DCCAKQEPER | |
| NECFLQHKDD NPNLPRLVRP EVDVMCTAFH DNEETFLKKY LYEIARRHPY | |
| FYAPELLEFA KRYKAAFTEC CQAADKAACL LPKLDELRDE GKASSAKQRL | |
| KCASLQKFGE RAFKAWAVAR LSQRFPKAEF AEVSKLVTDL TKVHTECCHG | |
| DLLECADDRA DLAKYICENQ DSISSKLKEC CEKFLLEKSH CIARVENDEM | |
| PADLPSLAAD FVESKDVCKN YAEAKDVFLG MFLYEYARRH PDYSVVLLLR | |
| LAKTYETTLE KCCAAADPHE CYAKVFDEFK PLVEEPQNLI KQNCELFEQL | |
| GEYKFQNALL VRYTKKVPQV STPTLVEVSR NLGKVGSKCC KHPEAKRMPC | |
| AEDYLSVVLN QLCVLHEKTP VSDRVTKCCT ESLVNRRPCF SALEVDETYV | |
| PKEFNAETFT FHADICTLSE KERQIKKQTA LVELVKHKPK ATKEQLKAVM | |
| DDFAAFVEKC CKADDKETCF AEEGKKLVAA SQAALGL | |
| DAHKSEVAHR FKDLGEENFK ALVLIAFAQY LQQCPFEDHV KLVNEVTEFA | SEQ ID NO:18 |
| KTCVADESAE NCDKSLHTLF GDKLCTVATL RETYGEMADC CAKQEPERNE | |
| CFLQHKDDNP NLPRLVRPEV DVMCTAFHDN EETFLKKYLY EIARRHPYPY | |
| APELLFFAKR YKAAFTECCQ AADKAACLLP KLDELRDEGK ASSAKQRLKC | |
| ASLQKFGERA FKAWAVARLS QRFPKAEFAE VSKLVTDLTK VHTECCHGDL | |
| LECADDPADL AKYICENQDS ISSKLKECCE KPLLEKSHCI AEVENDEMPA | |
| DLPSLAADFV ESKDVCKNYA EAKDVFLGMF LYEYARRHPD YSVVLLLRLA | |
| KTYETTLEKC CAAADPHECY AKVFDEFKPL VEEPQNLIKQ NCELFEQLGE | |
| YKFQNALLVR YTKKVPQVST PTLVEVSRNL GKVGSKCCKH PEAKRMPCAE | |
| DYLSVVLNQL CVLHEKTPVS DRVTKCCTES LVNRRPCFSA LEVDETYVPK | |
| EFNAETFTFH ADICTLSEKE RQIKKQTALV ELVKHKPKAT KEQLKAVMDD | |
| FAAFVEKCCK ADDKETCFAE EGKKLVAASQ AALGLDAGGG GSCGGGEGGG | |
| GSKTCYEGNG HFYRGKASTD TMGRPCLPWN SATVLQQTYH AHRSNALQLG | |
| LGKHNYCRNP DNRRRPWCYV QVGLKPLVQE CMVHDCAD | |
| EPRGPTIKPCPPCKCPAPNLLGGPSVFIFPPKIKDVLMISLSPIVTCVVVDVSED | SEQ ID NO:19 |
| DPDVQISWFVNNVEVHTAQTQTHREDYNSTLRVVSALPIQHQDWMSCKEFKCKVN | |
| NKDLPAPIERTISKPKGSVRAPQVYVLPPPEEEMTKKQVTLTCMVTDFMPEDIYV | |
| EWTNNGKTELNYKNTEPVLDSDGSYFMYSKLRVEKKNWVERNSYSCSVVHEGLHN | |
| HHTTKSFSRTPGK | |
| ARLEPRGPTI KPCPPCKCPA PNLLGGPSVF IFPPKIKDVL MISLSPIVTC | SEQ ID NO:20 |
| VVVDVSEDDP DVQISWFVNN VEVHTAQTQT HREDYNSTLR VVSALPIQHQ | |
| DWMSGKEFKC KVNNKDLPAP IERTISKPKG SVRAPQVYVL PPPEEEMTKK | |
| QVTLTCMVTD EMPEDIYVEW TNNGKTELNY KNTEPVLDSD GSYFMYSKLR | |
| VEKKNWVERN SYSCSVVHEG LHNHHTTKSF SRTPGKKTCY EGNGHFYRGK | |
| ASTDTMGRPC LPWNSATVLQ QTYHAHRSNA LQLGLGKHNY CRNPDNRRRP | |
| WCYVQVGLKP LVQECMVHDC AD | |
| AKTCYEGNGH FYRGKASTDT MGRPCLPWNS ATVLQQTYHA HRSNALQLGL | SEQ ID NO:21 |
| GKHNYCRNPD NRRRPWCYVQ VGLKPLVQEC MVHDCADRLE PRGPTIKPCP | |
| PCKCPAPNLL GGPSVFIFPP KIKDVLMISL SPIVTCVVVD VSEDDPDVQI | |
| SWFVNNVEVH TAQTQTHRED YNSTLRVVSA LPIQHQDWMS GKEFKCKVNN | |
| KDLPAPIERT ISKPKGSVRA PQVYVLPPPE EEMTKKQVTL TCMVTDFMPE | |
| DIYVEWTNNG KTELNYKNTE PVLDSDGSYF MYSKLRVEKK NWVERNSYSC | |
| SVVHEGLHNH HTTKSFSRTP GK | |

The cDNA sequence can be obtained from GenBank or a number of available sources. PCR based methods can be used to retrieve the cDNA from an appropriate library. The cDNA can then be conveniently stored in a vector such as the pGEM or pGEX vectors by standard ligation or plasmid manipulation methods. The polypeptide encoding regions are then transferred into an appropriate, selected expression cassette or vector. Specific examples of vectors for various applications exist, including gene therapy (Chen et al., Hum Gen Ther 11: 1983-96 (2000); MacDonald et al., Biochecm Biophys Res Comm 264:469-477 (1999); Cao et al., J Biol Chem 271:29461-67 (1996); Li et al., Hum Gene Ther 10:3045-53 (1999)). For the examples that follow, the method of Soubrier et al., Gene Therapy 6:1482-1488 (1999), is used to prepare recombinant adenovirus with E1/E3 deletion, CMV expression promotor and SV40 polyA. The plasmid vector used below contains the Amp resistance gene, the CMV promotor, the SV40 poly A sequence, and the IL-2 signal sequence for efficient secretion. The fairly robust adenoviral system can be selected for its ability to be used in a variety of cell types, whereas the plasmid system is selected for its relative efficiency of vector introduction. One skilled in the art is familiar with selecting or modifying vectors with these or other elements for use.

Once cloned and inserted into an appropriate vector, any of the abrogen encoding sequences or abrogen derivatives encoding sequences can be assayed for specific activity related to anti-angiogenesis using the Examples below or an assay mentioned here or in the references.

In a preferred embodiment for expressing a recombinant abrogen polypeptide, a vector comprising the coding region for human serum albumin linked to the C-terminus of the abrogen encoding region is used (see, for example, Lu et al., FEBS Lett. 356: 56-9 (1994)). Other fusion proteins or chimeric proteins can also be used. In another embodiment of a fusion protein, the abrogen encoding region is linked to an immunogenic peptide or polypeptide encoding region. These fusions can be used in created antibodies or monoclonal antibodies against an abrogen. Methods for preparing antibodies are well known in the art and both the purified abrogen polypeptides and fusion of them can be used to prepare antibodies. Monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, such procedures involve immunizing an animal (preferably a mouse) with polypeptide or, more preferably, with a secreted polypeptide expressing cell. The mice splenocytes are extracted and fused with a suitable myeloma cell line, such myeloma cell line SP20, available from the ATCC. After fusion, the resulting hybridoma cells are selectively maintained in HAT medium and then cloned by limiting dilution as described (Wands et al., Gastroenterology 80:225-232 (1981)). The hybridoma cells obtained through such a selection are then assayed to identify clones, which secrete antibodies capable of binding the polypeptide. Additional fusions can be used to ease purification of abrogen polypeptides, including poly-His tracks, constant domain of immunoglobulins (IgG), the carboxy terminus of either Myc or Flag epitope (Kodak), and glutathione-S-transferase (GST) fusions. Plasmids for this purpose are readily available.

A relatively simple method for preparing recombinant or purified abrogen polypeptide involves the baculovirus expression system or the pGEX system (Nesbit et al., Oncogene 18:6469-6476 (1999), Nesbit et al., J of Immunol 166:6483-90 (2001)). In the baculovirus system, plasmid DNA encoding the abrogen polypeptide is cotransfected with a commercially available, linearized baculovirus DNA (BaculoGold baculovirus DNA, Pharmingen, San Diego, Calif.), using the lipofection method (Felgner et al., PNAS 84:7413-7417 (1987)). BaculoGold virus DNA and the plasmid DNA are mixed in a sterile well of a microtiter plate containing 50 ul of serum-free Grace's medium (Life Technologies Inc., Gaithersburg, Md.). 10 μl Lipofectin and 90 μl Grace's medium are added, mixed and incubated for 15 minutes at room temperature. The transfection mixture is added drop-wise to Sf 9 insect cells (ATCC CRL 1711), and seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. The cells are cultured at 27° C. for four days. The cells can then be selected for appropriately transduction and assayed for the expression of abrogen polypeptide. If a fusion polypeptide was desired, the fusion polypeptide can be purified by known techniques and used to prepare monoclonal antibodies.

### Example 2

Proliferation Analysis of Transduced HUVEC Using Alamar Blue.

A number of different assays for analyzing cell proliferation, tubule formation, cell migration, endothelial cell growth, and tumor metastasis exist. Some of them are described in the references cited.

Human umbilical vein endothelial cells (HUVEC: Clonetics, San Diego) are seeded at 5×10⁵ cells/well of 6-well-plate in EGM-2 medium. The cells are incubated overnight at 37° C., 5% CO₂. Endothelial Cell Basal Medium (EBM) and Endothelial Cell Growth Medium (EGM) are available (Clonetics, San Diego). The medium is aspirated off and 500 ul of ECM medium containing 100 IT/cell viruses put over cells. The cells are incubated at 37° C. for 2 hours, then aspirated and 1.5 ml EGM-2 medium is added. The cells are again incubate overnight at 37° C.

The cells are trypsinized, counted, and seeded at 2000 cell/well of 96-well-plate in EGM-2 medium. The cells are incubated at 37° C. for 3 hours. The medium is changed into 200 μl of the following medium: Control=ECM+0.5% FBS; Test 1=control medium with bFGF 10 ng/ml; Test 2=control medium with VEGF 10 ng/ml; Test 3=control medium with bFGF 10 ng/ml+VEGF 10 ng/ml. After changing the medium, the cells are incubated at 37° C. for 5 days. 20 μl Alamar Blue (BioSource International) for each well is added. Plates are incubated at 37° C. for 6 hours and then the OD read at 570 nm and 595 nm.

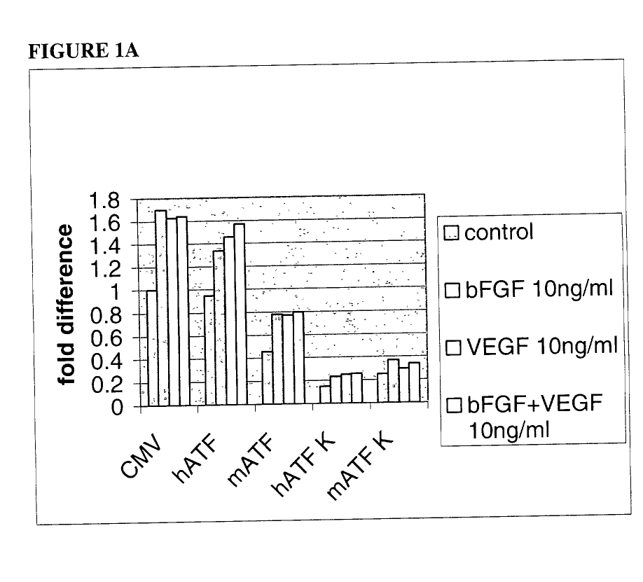

Typical results are depicted in FIG. 1. From the results of this proliferation assay, both human and mouse ATF-K polypeptides (SEQ ID NO.: 1, 3, 5, and 7) are very effective in abrogating the proliferation of endothelial cells induced by bFGF and VEGF.

### Example 3

Assay of Transduced HUVEC Embedded in Fibrin Gel.

In an assay that distinguishes the abrogen activity from angiostatin, human umbilical vein endothelial cells (HUVEC: Clonetics, San Diego) are seeded (passage 3, growing in EGM-2 medium) at 5×10⁵ cells/well of 6-well-plate in EGM-2 medium. The embedded cell assay also or alternatively provides data concerning the invasiveness of the endothelial cells in response to certain treatments. Endothelial cell tubule formation induced by pro-angiogenic factors such as FGF and VEGF, a characteristic measured by this assay, can be directly correlated to angiogenesis. The abrogen polypeptides used here can inhibit or reduce angiogenesis by inhibiting tubule formation. The use of virally transduced HUVEC can provide very detailed information as to the effects that a selected abrogen polypeptide or derivative has on primary cell types. The potential anti-angiogenic agents are introduced by transduction of the cells (m-ATF, h-ATF, m-ATF-K and h-ATF-K, CMV empty was included as a control) using a recombinant human adenovirus.

adenovirus VP/ml vp/IT IT/ml cell/flask ul/flask

Ad CMV 5.85E+12 100 5.85E+10 5.00E+068.55

Ad HATF 2.76E+12 100 2.76E+10 5.00E+0618.12

Ad mATF 5.00E+12 100 5.00E+10 5.00E+0610

Ad hATF-K 2.02E+12 161 1.25E+10 5.00E+065

Ad mATF-K 5.19E+12 51 1.02E+11 5.00E+0640

The fibrin gel includes PBS (control), VEGF or bFGF. HUVEC cells are split ½ to ⅓ the day before transduction. On the day of the transduction, the cells are washed with PBS. 10 ml of serum free medium containing 100:1 (IT: cell ratio) of virus is incubated with the HUVEC for 2 hours to transduce the cells. The medium is then removed and the cells washed with PBS and 20 ml of full HUVEC medium placed in each T150 flask.

48 hours following transduction the cells are trypsinized and the concentration of each cell solution adjusted to 5×10⁵ cell/ml. The assay is performed in a 24 well plate. Each well is coated with 200 μl of fibrinogen solution (12 mg/ml) and 8 ul of thrombin (50 U/ml). Then in each well is added (according to the conditions):

VEGF165 (2 μl ), b-FGF(2 μl) or nothing (final [growth factor]=1 ug/ml)

Thrombin (20 ul) of a 1000 U/ml solution.

250 μl cell solution for a final concentration of 5×105 cells/ml

250 μl of fibrinogen

Gels set in about 30 seconds. Then, 1.5 ml of medium is added on top. Each type of infected cells was assayed with VEGF165 alone, b-FGF alone or without any growth factor other than those already present in the medium.

After 6 days medium is removed and cells subjected to staining with Dif-Quick for enhanced visualization under microscopy. Fibrin plugs are fixed in 10% formalin, and then subjected to the 3 Dif Quick stains for 15 mins each before being rinsed in PBS and then fixed with 10% formalin again.

Representative photographs of cells are depicted in FIG. 1B. Tubules can be seen in control cells, whereas no tubules are detected in the hATF-K and mATF-K transduced cells. Tubule formation can be correlated with endothelial cell invasiveness, a characteristic of angiogenic activity. Thus, the lack of tubule formation in the abrogen polypeptide samples (human ATF Kringle and mouse ATF Kringle) demonstrate an inhibtion of endothelial cell invasiveness, correlating to an inhibition of angiogenesis and metastasis. In the FIG. 1B pictures, transduced HUVEC are treated with control PBS, bFGF, or VEGF, which give the following results. For CMV control: limited structure is visible when PBS is in the fibrin gel; with VEGF there is robust proliferation showing the phenotype generated; tubules are clearly visible and are ubiquitous throughout the gel, some extensions are quite long; in the presence of bFGF the response is not as robust, the structures, which form, are long and spindle like in appearance. For full human ATF polypeptide: in PBS there are a considerable number of structures formed; the response is far more than that seen with control CMV transduced endothelial cells, also in relation to the CMV control there has been a robust response with the addition of bFGF, which is definitely synergistic with the human ATF transduced cells in comparison to those transduced with CMV; in the presence of VEGF there has been a considerable drop in the number of visible structure when compared to the CMV transduced cells. For full mouse ATF polypeptide: regardless of condition there are no structures forming in any of the gels. For human ATK Kringle (abrogen of SEQ ID NO.: 1): regardless of condition there are no structures forming in any of the gels. For mouse ATF Kringle (abrogen of SEQ ID NO.: 3): regardless of condition there are no tubule structures forming in any of the fibrin gels.

Without limiting the scope of the invention to any particular mode of action or mechanism, applicants offer the following possible explanation of these results. Human ATF still has the EGF like growth factor domain and may stimulate the growth of endothelial cells, which are human in origin. This growth is potentiated in the presence of ubiquitous bFGF in this assay, as one of the downstream effects of bFGF is the upregulation of uPAR. This synergy is observed when cells are transduced with human ATF in the presence of bFGF. In the absence of bFGF, human ATF can stimulate low level uPAR and presumably inhibits growth through the action of the kringle. Hence the observed decrease in number of structures when compared to CMV control. Mouse ATF does not cross react with human uPAR. Therefore, the mode of action is mediated through the kringle domain. With human and mouse ATF-K, there is no growth factor domain so no proliferative events can be initiated. This is specific to both bFGF and VEGF induced proliferative responses.

### Example 4

In Vivo Expression of Abrogen Polypeptides Using Adenoviral Vectors.

For in vivo documentation of the activity of abrogen, a first experiment involves the systemic injection iv of 1×1011 VP of hATF-K expressing adenovirus. Circulating levels of hATF-K as shown by Western can be measured. Exemplary expression levels at d4 can be between 500-1000 ng/ml in either SCID or SCID/Beige mice. The 4T1 spontaneously metastatic breast cell line in SCID mice is used in which animals are injected with 2×105 cells sub-cutaneously in the right flank. At d7, when tumors were 20-40 mm3, adenovirus is injected at 1×1011 vp: Tris, CMV1.0 control Ad; MATF-K; and HATF-K. A second and third iv administration of adenovirus can be performed. Lung metastasis is then measured at about day 35, as described below.

### Example 5

In Vivo Expression of Abrogen Polypeptides Using Plasmid Vectors.

Two tumor models are used, employing 4T1 tumor cells and 3LL Boston tumor cells. In the assay, the anti-tumor activity of abrogen polypeptide in the prophylactic murine Lewis lung carcinoma model, 3LL-B, in C57BL/6 mice is tested. The assay is designed to assess whether circulating levels of abrogen prevent and/or reduce the formation and growth of spontaneously formed metastases from subcutaneously implanted primary tumors. The tumor cells are cultured in DMEM containing 10% FCS, sodium pyruvate, nonessential amino acids, Pen-Strep, and L-Glutamine until prepared for injection using a buffered saline solution. The tumor cells are injected into the right flank of 8-10 week old C57BL/6 or BALB/c female mice via subcutaneous injection of a suspension of 2.5×105 tumor cells. Six days prior to tumor cell injection, the 25 ul of the plasmid solutions (25ug DNA in Tris EDTA with 10% glycerol) are injected into the tibialis cranialus muscle. The injection site is then exposed to 4 pulses (1 pulse per second) at 100 mV using a square wave pulse generator (the electrotransfer method, ET). Alternatively, the electrotransfer enhancement can utilize four electric pulses of 100 V (250 V/cm) at 1 Hz with a pulse length of 20 msec. On about day 15 post cell injection, the primary subcutaneous tumor was surgically removed. At day 35, the lungs are collected and tumor nodules measured. Expression levels are measured on day—1, 7, and 14 relative to electrotransfer. A control alkaline phosphatase expressing plasmid (mSEAP) is used to assay expression.

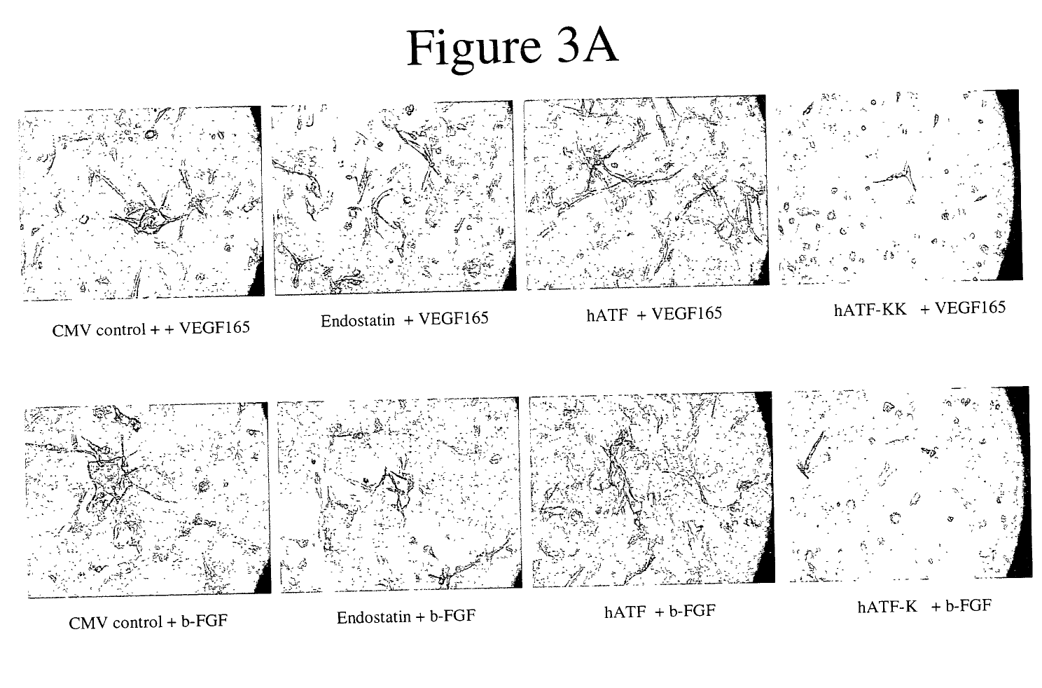

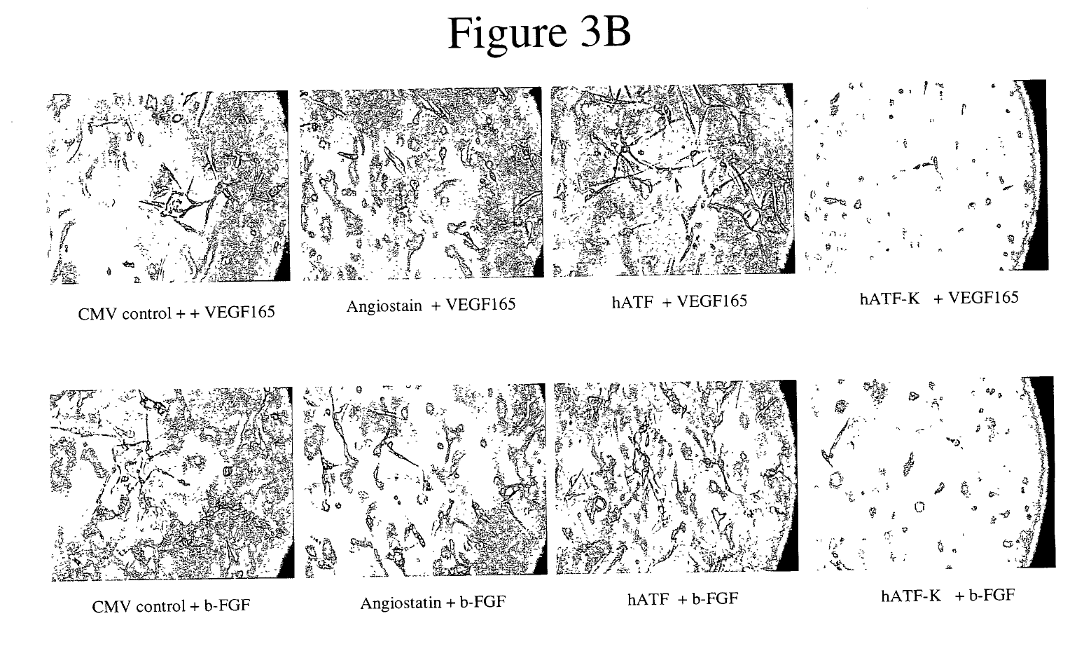

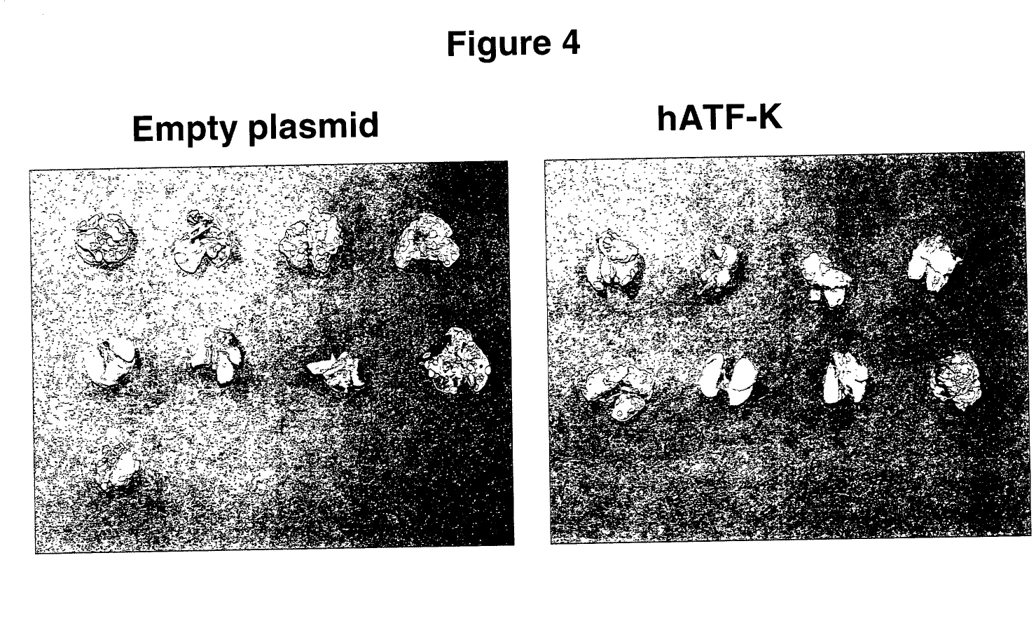

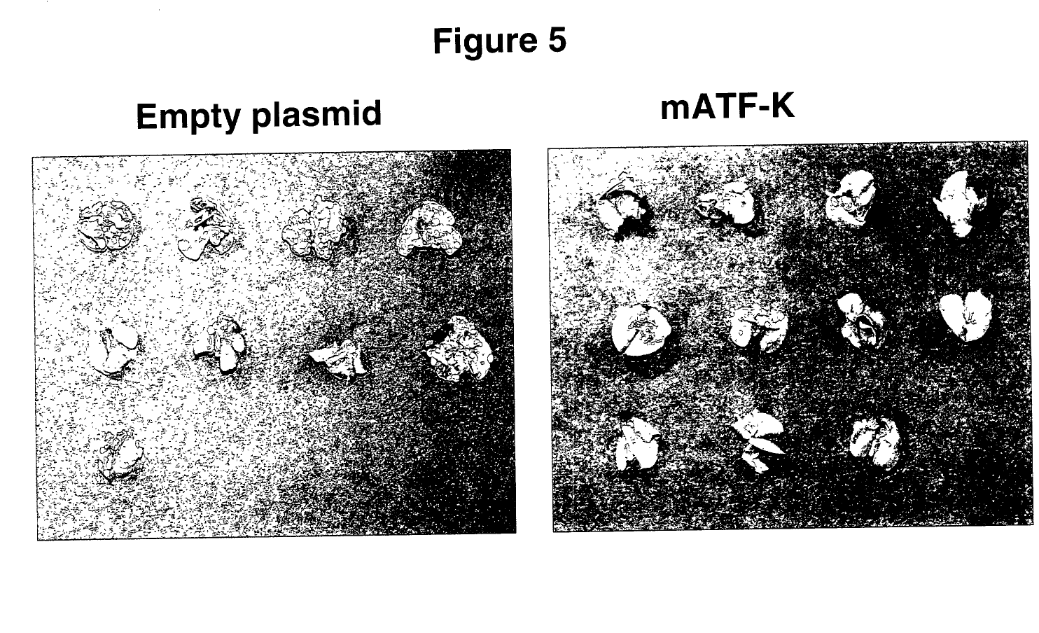

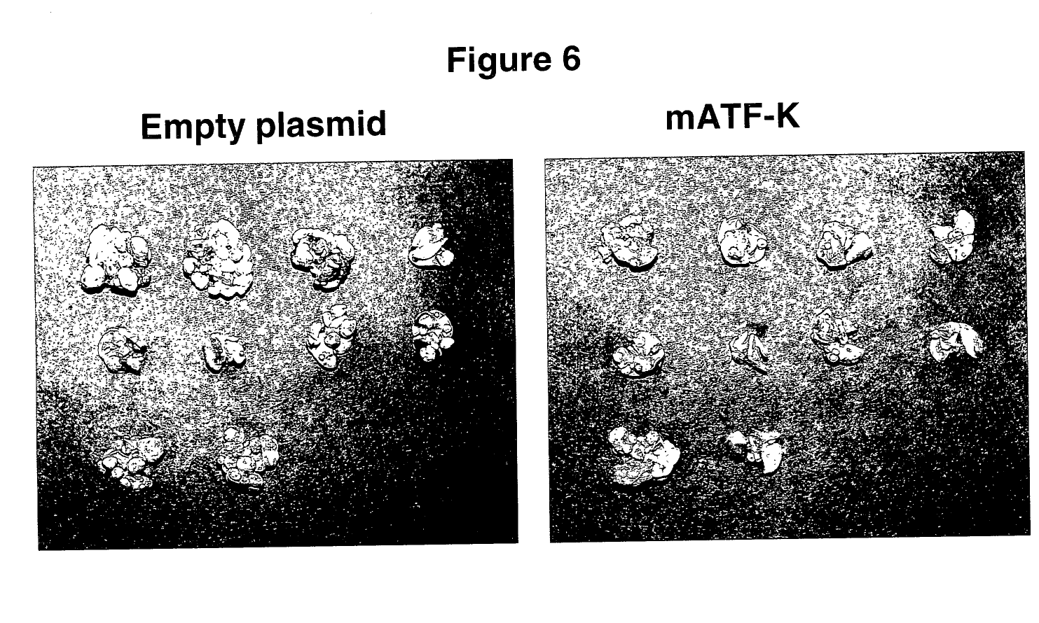

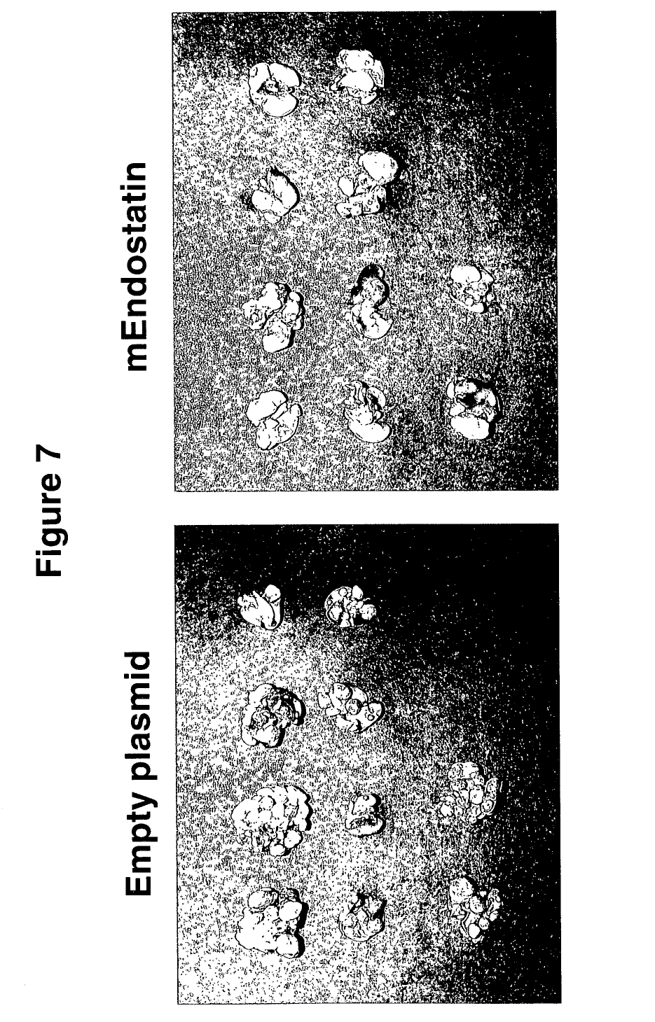

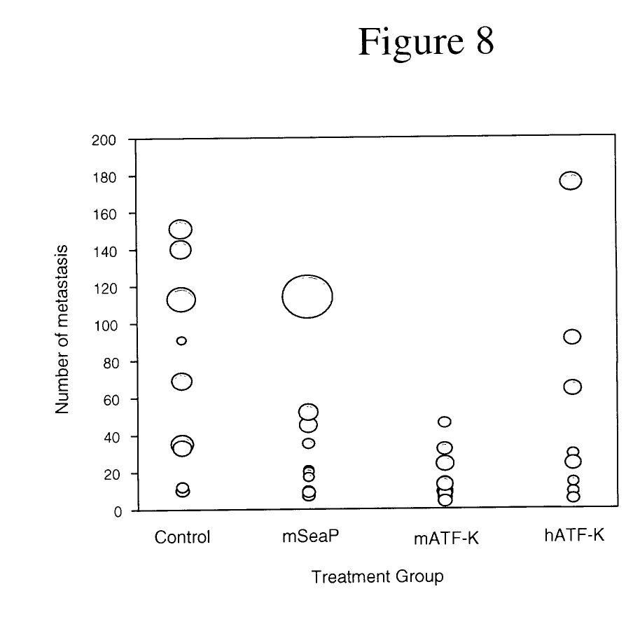

The results of one set of experiments are depicted in FIGS. 4-10. The empty expression plasmid and the mSEAP control plasmid treatments resulted in many lung tumor nodules. In both the 4T1 and 3LL tumor models, the mATF-K and hATF-K abrogen polypeptides reduced the size and number of metastasis. The reduction in size and number is at least equivalent to those of the known anti-angiogenic polypeptides endostatin and angiostatin (FIG. 10).

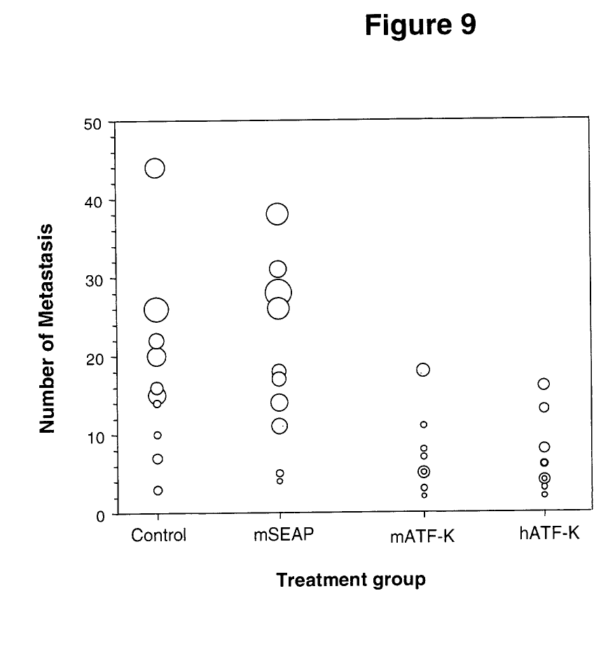

Another set of assays with 3-LL Boston cells employing electrotransfer enhancement with four electric pulses of 100 V (250 V/cm) at 1 Hz with a pulse length of 20 msec are shown in FIG. 11. Metastases were counted using a dissecting microscope. The FIG. 11 pictures of the lungs show that the formation of spontaneous lung metastases from the primary subcutaneous tumor was significantly reduced in the two therapeutic groups receiving plasmid DNA encoding either mouse of human ATF Kringle (listed as MuPAK or HuPAK here). Lung metastases counts as well as lung weights, reflected by the diameter of the “bubble” in panel C, were reduced in both treatment groups. Delivery of plasmid DNA encoding either the murine secreted alkaline phosphatase (mSEAP) or no protein as control to the T. cranialis muscle did not result in a significant reduction of lung metastases. Similar results can be obtained in the prophylactic 4T1 mammary tumor model (data not shown).

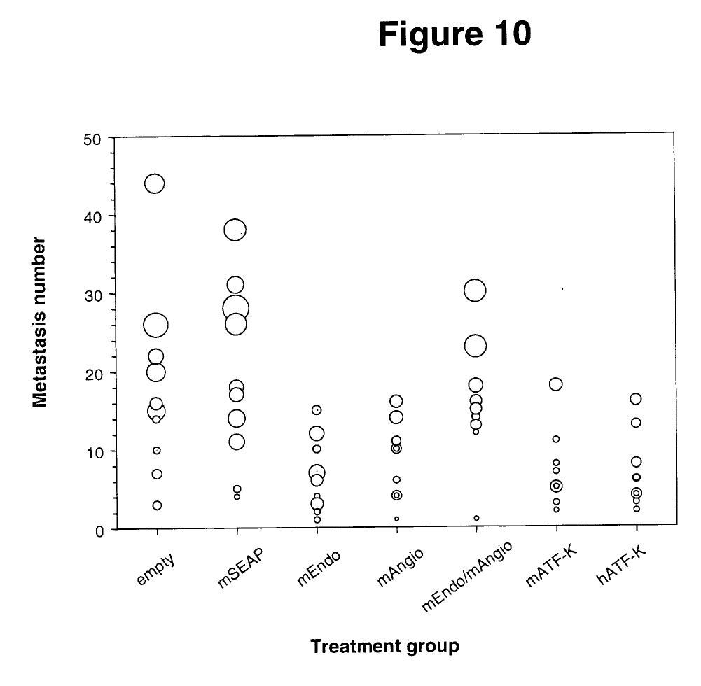

To assess the anti-tumor activity of systemically expressed abrogen polypeptides in a human breast adenocarcinoma xenograft model of SCID/bg mice, MDA-MB-435 tumor cells are used. These cells are significantly less aggressive as compared to the 4T1 and 3LL-B syngeneic mouse tumor models. However, spontaneous lung metastases formation is established in the time frame of 35 days post subcutaneous cell injection. Subcutaneous palpable MDA-MB-435 tumors are established by injecting SCID/bg mice with 10⁶ tumor cells. On day 10 post injection, plasmid DNA was transferred to the Tibialis cranialis muscle using electrotransfer as described previously. Briefly, 25 μg of plasmid DNA (a total of 50 μg) in a 25 μl volume are injected directly into each T. cranialis muscle followed by four electric pulses of 100 V (250 V/cm) at 1 Hz with a pulse length of 20 msec. The primary tumor is carefully removed when the volume reached between 250 and 350 mm3, i.e. on day 39 or 44 post cell injections depending on the growth of the primary tumor. The study is terminated on day 89 and lungs harvested carefully and fixed in Bouin's solution. Metastases are counted using a dissecting microscope. FIG. 12 shows pictures of the lungs.

Lungs from mice treated with either mouse or human AFT-Kringle containing polypeptide, FIG. 12 panels B and C, bear significantly fewer metastases compared to the control group (panel A) treated with the plasmid encoding mSEAP. Overall lung metastases counts are significantly reduced as shown in panel D. By the time of treatment at day 10, no lung metastases have been formed in the lung of SCID/bg mice, so it is most likely that the systemic expression of abrogen from the muscle prevents the formation and/or growth of distant lung metastases from the primary subcutaneous tumor. This demonstrates an inhibition of angiogenesis, a hallmark for the growth of metastatic tumors.

### Example 6

Production of Derivative Abrogen Polypeptides by PCR Based Site-Directed Mutagenesis.

In one method for generating an abrogen derivative, four oligonucleotide primers are used. Two of these are primers that flank the ends of the cDNA (SEQ ID NO.: 2, 4, 6, or 8 ) and contain convenient restriction sites for cloning into a desired vector. The other two mutagenic primers are complementary and contain the mutation(s) of interest. Typically, the mutagenic primers overlap by about 24 base pairs. Two separate PCR reactions are performed, each using a different outside primer and a different mutagenic primer that anneal to opposite strands of the DNA template. The amplified product from both PCR reactions are purified and added to a new primeness PCR mix.

After a few PCR cycles, the two products are annealed and extended at the region of overlap yielding the derivative product. The two outside primers are then added to this mixture to amplify the cDNA product by PCR. This method can be used to introduce amino acid substitutions at any point in an abrogen sequence.

In addition to the conservative amino acid substitutions noted throughout the disclosure, one skilled in the art is familiar with numerous methods for analyzing and selecting homologs and derivative sequences to use as abrogen sequences. For example, the sequence identified as “Putative-K1 (Est)” in FIG. 2 can be identified by searching for homologs using GenBank, an EST database, or any cDNA or genomic DNA database available. The EST can be pulled from a library, PCR amplified using primers specific for the EST, or synthesized using automated methods. Once isolated, the polypeptide encoding region can be cloned into an appropriate vector and tested as described above.

### Example 7

Construction of IL2sp-abrogen Polypeptide

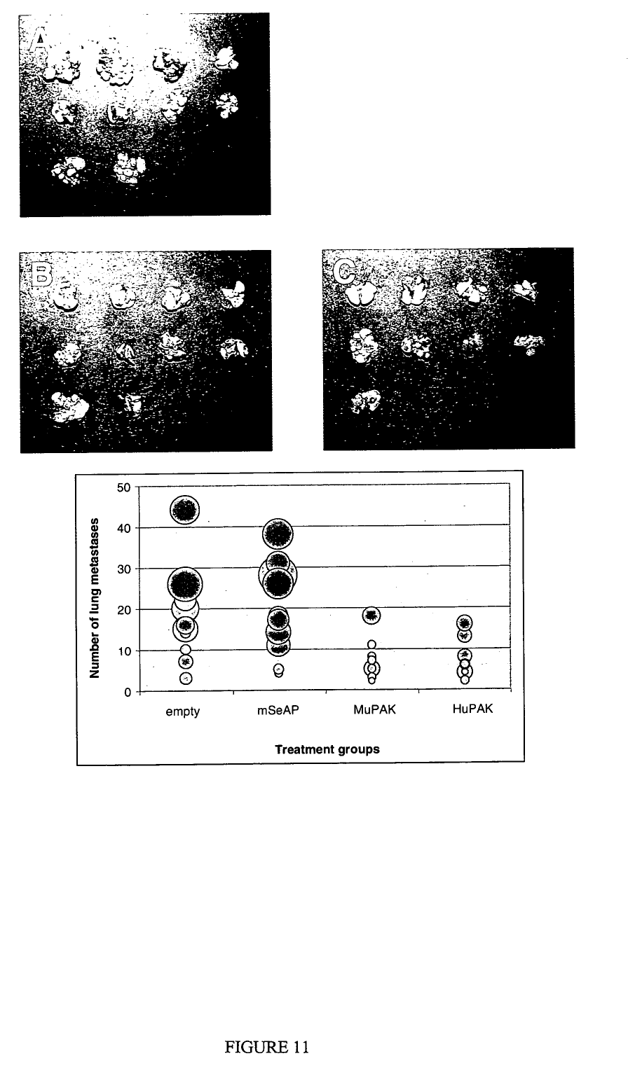

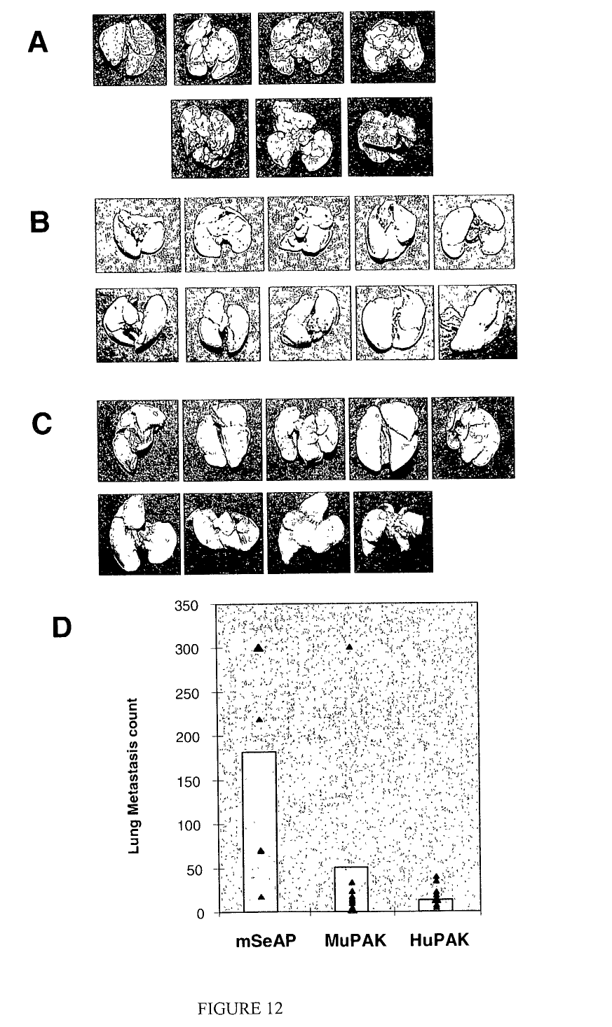

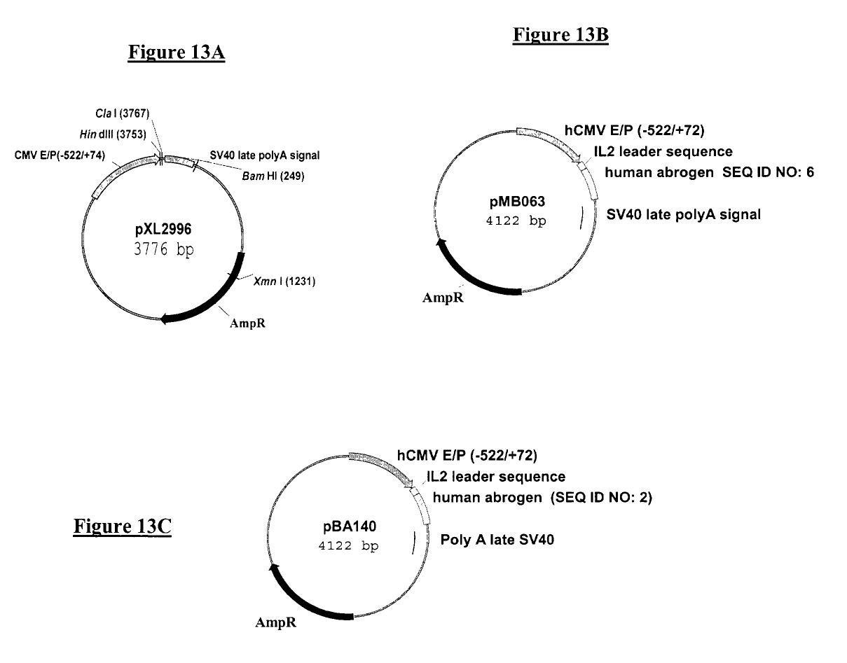

The combined techniques of site-directed mutagenesis and PCR amplification allowed to construct a chimeric gene encoding a chimeric peptide resulting from the translational coupling between the first 20 amino acids of the interleukin 2 signal peptide, which represent a signal sequence or signal peptide that is cleaved to produce the mature factor (Tadatsugu, T. et al. (1983) Nature 302:305) and the abrogen sequences as set forth in SEQ ID NO: 4 (IL2sp-abrogen). These hybrid genes were preferably bordered in 5′ of the translational initiator ATG and in 3′ of the translational stop codon and encode chimeric proteins of the IL2sp-abrogen. The hybrid gene is cloned in the pXL2996 (FIG. 13A), under the control of the human CMV Enhancer/promoter (−522/+72) and upstream of a SV40 late poly A signal. The resulting plasmid pMB063 as described in FIG. 13A was obtained. The abrogen peptide secreted from the plasmid pMB063 retained an alanine from the IL-2 signal peptide at the N-terminus, and thus contains a 87 amino acid sequence as set forth in SEQ ID NO: 9.

The hybrid nucleotide sequence comprising the interleukine 2 signal peptide sequence and the abrogen sequence as set forth in SEQ ID NO: 2 was cloned in plasmid pXL 2996 downstream of the human CMV enhancer/promoter (−522/+72) and upstream of a SV40 late poly A signal. The resulting plasmid pBA140 as described in FIG. 13B was obtained. The abrogen peptide secreted from the plasmid pBA140 also retained an alanine from the IL-2 signal peptide at the N-terminus, and thus contains a 87 amino acid sequence as set forth in SEQ ID NO: 10.

### EXAMPLE 8

Construction of Fusion Proteins of Abrogen and HSA

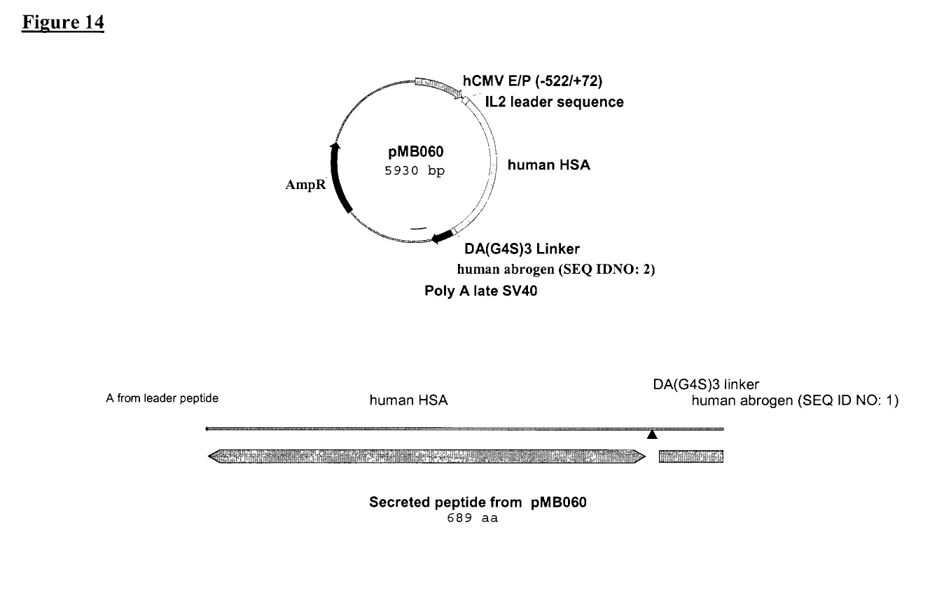

A nucleotide fragment containing from 5′ to 3′ the IL-2 signal peptide, the nucleotide sequence encoding the human HSA as set forth in SEQ ID NO: 11, a linker, and the abrogen sequence as set forth in SEQ ID NO: 2 was cloned in plasmid pXL2996 downstream to the human CMV promoter and upstream of a SV40 polyA. The linker DA(G₄S)₃ was used (SEQ ID NO: 12). The construct of the fusion protein IL2sp-HSA-linker-abrogen and the resulting plasmid designated pMB060 are shown in FIG. 14. The fusion protein HSA/abrogen secreted from the plasmid pMB060 has the sequence as set forth in SEQ ID NO: 13.

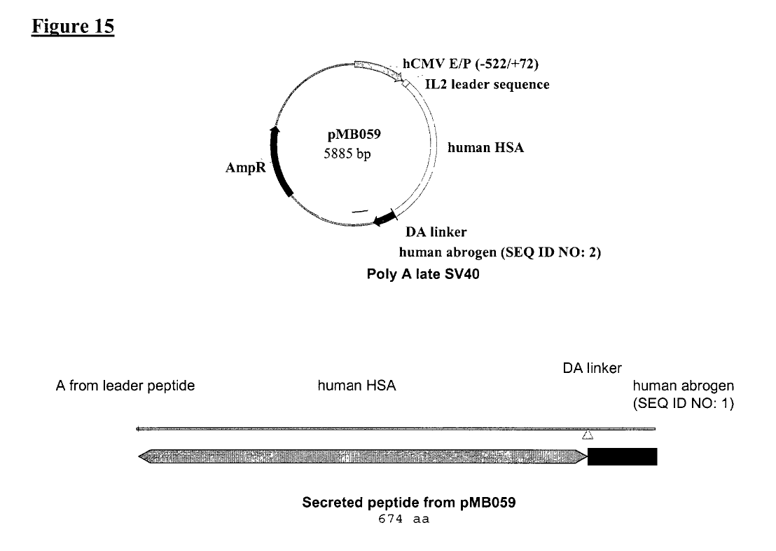

Another linker DA (Asp-Ala) was used. The chimeric construct of the fusion protein IL2sp-HSA-DA linker-abrogen and the resulting plasmid is designated pMB059 are displayed in FIG. 15. The fusion protein HSA/abrogen secreted from the plasmid pMB059 has the sequence as set forth in SEQ ID NO: 14.

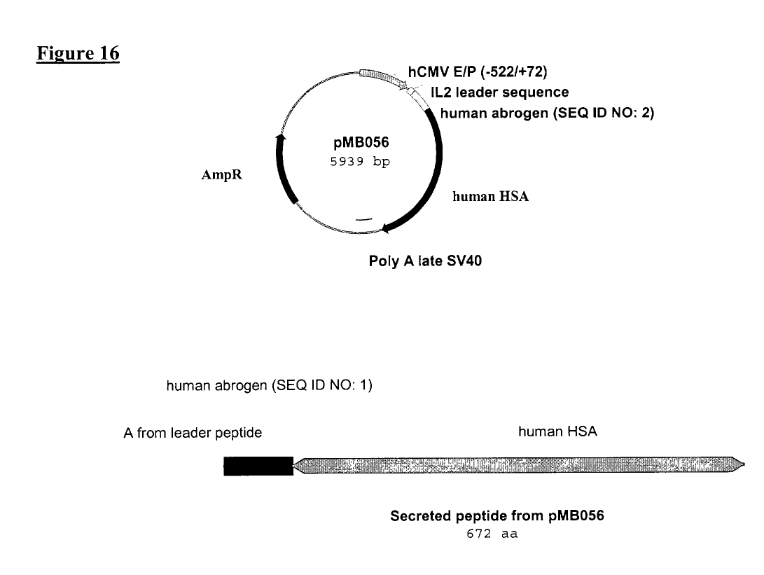

A nucleotide fragment containing from 5′ to 3′ the IL-2 signal peptide, the abrogen nucleotide sequence as set forth in SEQ ID NO: 2, and the sequence of the human HSA (SEQ ID NO: 11), was cloned in pXL2996 downstream to the human CMV promoter and upstream of a SV40 polyA. The resulting plasmid is designated pMB056 and construct are displayed in FIG. 16. The fusion protein HSA/abrogen secreted from the plasmid pMB056 has the sequence as set forth in SEQ ID NO: 15.

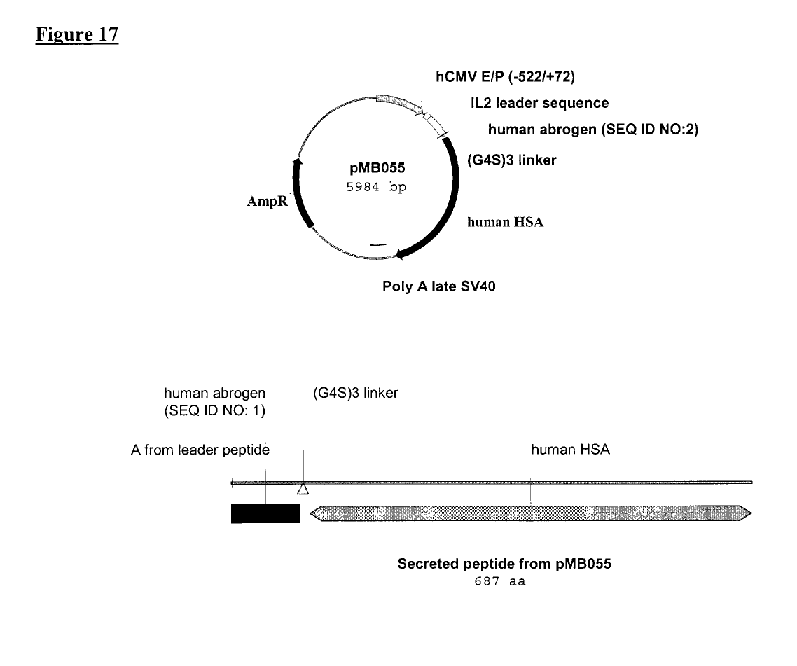

A nucleotide fragment containing from 5′ to 3′ the IL-2 signal peptide, the abrogen nucleotide sequence having the sequence as set forth in SEQ ID NO: 2, a (G₄S)₃ linker (as set forth in SEQ ID NO: 16) and the sequence of the human HSA, was cloned downstream to the human CMV promoter and upstream of a SV40 polyA. The chimeric construct of the fusion protein IL2sp-abrogen-linker-HSA and the resulting plasmid designated pMB055 are displayed in FIG. 17. The fusion protein abrogen/HSA secreted from the plasmid pMB055 has the sequence as set forth in SEQ ID NO: 17.

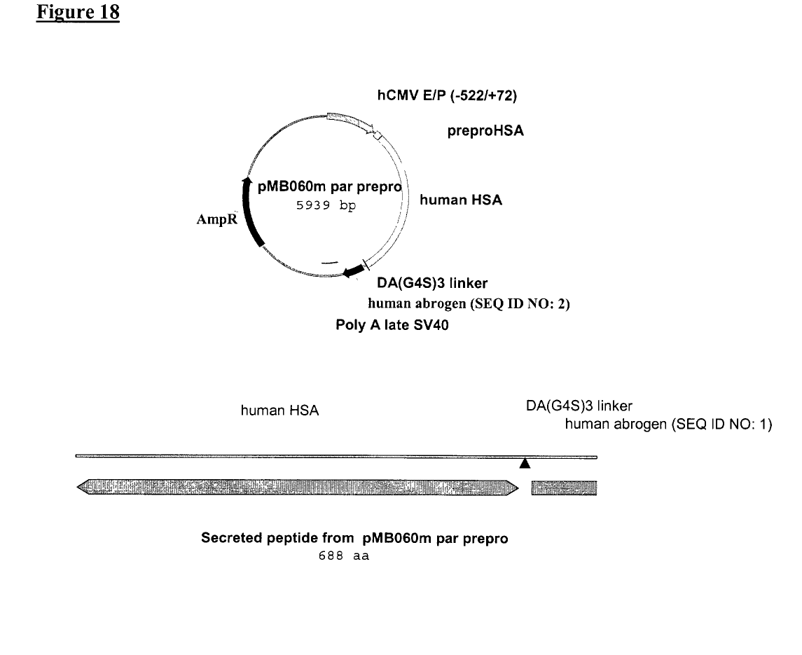

Alternatively, a nucleotide sequence containing from 5′ to 3′ the prepro signal of HSA, the human HSA, a sequence encoding a DA(G₄S)₃ linker and the abrogen nucleotide sequence as set forth in SEQ ID NO: 2 was cloned in the plasmid pXL2996 downstream to the human CMV promoter and upstream of a SV40 polyA. The resulting plasmid is designated pMB060m and the fusion protein prepro HSA—human HSA—DA(G₄S)₃ linker-abrogen are displayed in FIG. 18. The fusion protein HSA/abrogen secreted from the plasmid pMB060m has the sequence as set forth in SEQ ID NO: 18.

### Example 10

Construction of Fusion Proteins of Abrogen and IgG2a

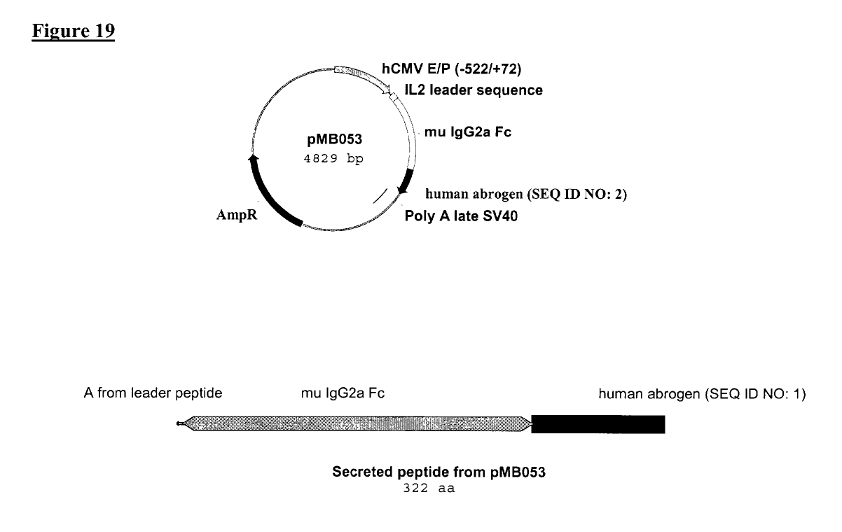

A nucleotide fragment containing from 5′ to 3′ the IL-2 signal peptide, the murin IgG2a Fc region (SEQ ID NO: 19) and the human abrogen nucleotide sequence having the sequence as set forth in SEQ ID NO: 2 was cloned in pXL2996 downstream to the human CMV promoter and upstream of a SV40 polyA. The resulting plasmid is designated pMB053 and the fusion construct are displayed in FIG. 19. The fusion protein IgG2a/abrogen secreted from the plasmid pMB053 has the sequence as set forth in SEQ ID NO: 20.

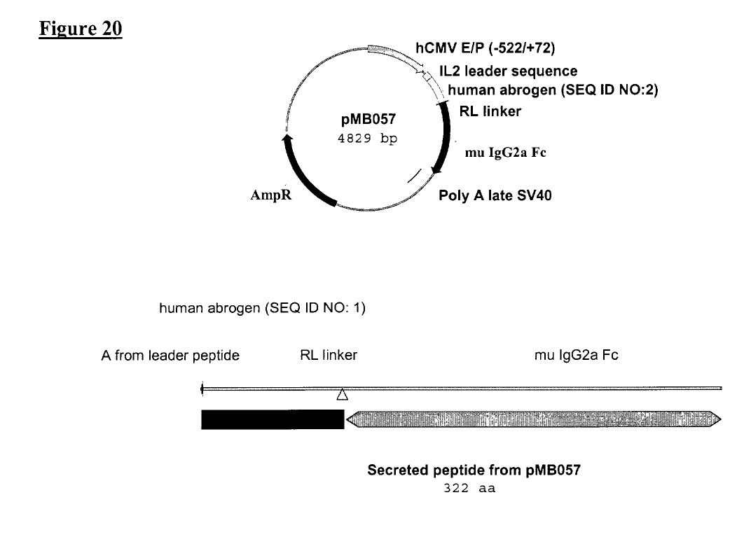

A nucleotide fragment containing from 5′ to 3′ the IL-2 signal peptide, the human abrogen nucleotide sequence having the sequence as set forth in SEQ ID NO: 2, the nucleotide sequence coding for a RL (Arginine-Leucine) linker, the murin (mu) IgG2a Fe region was cloned in pXL2996 downstream to the human CMV promoter and upstream of a SV40 polyA. The resulting plasmid is designated pMB057 and the fusion construct are shown in FIG. 20. The fusion protein abrogen/IgG2a secreted from the plasmid pMB057 has the sequence as set forth in SEQ ID NO: 21.

### Example 11

Construction of Plasmids Suitable for the Production of Recombinant Abrogen or Fusion Polypeptide

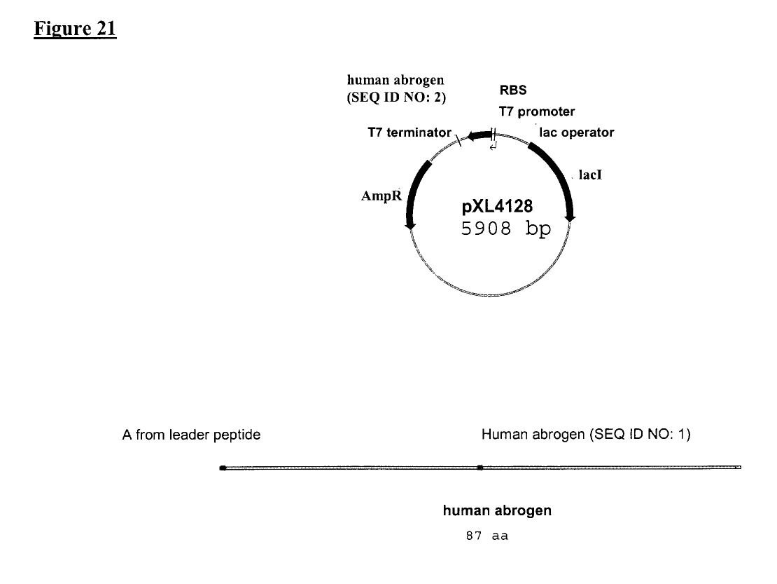

The plasmid pXL4128, which is represented in FIG. 21 and comprises the bacteriophage T7 promoter was also constructed, and is suitable for the production of the abrogen peptide in E coli. Such plasmid for the production in E.coli are also described in U.S. Pat. No. 6,143,518. The plasmid pYG404 as described in the Patent application EP 361 991, which comprise the sequence encoding the prepro-HSA gene may be used. For example, the C-terminal of HSA is coupled in transitional phase with a linker sequence and the abrogen nucleotide sequence. The resulting plasmid is used for production of the peptide in yeasts, for example.

References:

The references cited below may be referred to above by the reference number. Each of the references is specifically incorporate herein by reference.

1. Andreasen, P. A., et al., The urokinase-type plasminogen activator system in cancer metastasis: a review. Int J Cancer, 1997. 72(1): p. 1-22.

2. Mukhina, S., et al., The chemotactic action of urokinase on smooth muscle cells is dependent on its kringle domain. Characterization of interactions and contribution to chemotaxis. J Biol Chem, 2000. 275(22): p. 16450-8.

3. Rabbani, S. A., et al., Structural requirements for the growth factor activity of the amino-terminal domain of urokinase. J Biol Chem, 1992. 267(20): p. 14151-6.

4. Quax, P. H., et al., Binding of human urokinase-type plasminogen activator to its receptor: residues involved in species specificity and binding. Arterioscler Thromb Vase Biol, 1998. 18(5): p. 693-701.

5. Min, H. Y., et al., Urokinase receptor antagonists inhibit angiogenesis and primary tumor growth in syngeneic mice. Cancer Res, 1996. 56(10): p. 2428-33.

6. Li, H., et al., Systemic delivery of antiangiogenic adenovirus AdmATF induces liver resistance to metastasis andprolongs survival of mice. Hum Gene Ther, 1999. 10(18): p. 3045-53.

7. Tang, H., et al., The urokinase-type plasminogen activator receptor mediates tyrosine phosphorylation of focal adhesion proteins and activation of mitogenactivated protein kinase in cultured endothelial cells. J Biol Chem, 1998. 273(29): p. 18268-72.

8. Soff, G. A., Angiostatin and angiostatin-related proteins. Cancer Metastasis Rev, 2000. 19(1-2): p. 97-107.

9. Kleiner, D. E., Jr. and W. G. Stetler-Stevenson, Structural biochemistry and activation of matrix metalloproteases. Curr Opin Cell Biol, 1993. 5(5): p. 891-7.

10. Aguirre Ghiso, J. A., et al., Deregulation of the signaling pathways controlling urokinase production. Its relationship with the invasive phenotype. Eur J Biochem, 1999. 263(2): p. 295-304.

11. Dong, Z., et al., Macrophage-derived metalloelastase is responsible for the generation of angiostatin in Lewis lung carcinoma. Cell, 1997. 88(6): p. 801-10.

12. Cao, Y., et al., Kringle domains of human angiostatin. Characterization of theanti-proliferative activity on endothelial cells. J Biol Chem, 1996. 271(46): p. 29461-7.

13. Cao, Y., et al., Kringle 5 of plasminogen is a novel inhibitor of endothelial cell growth. J Biol Chem, 1997. 272(36): p. 22924-8.

14. Nesbit, M., Abrogation of tumor vasculature using gene therapy. Cancer Metastasis Rev, 2000. 19(1-2): p. 45-9.

15. Lee, T. H., T. Rhim, and S. S. Kim, Prothrombin kringle-2 domain has a growth inhibitory activity against basic fibroblast growth factor-stimulated capillary endothelial cells. J Biol Chem, 1998. 273(44): p. 28805-12.

16. Rhim, T. Y., et al., Human prothrombin fragment 1 and 2 inhibit bFGF-induced BCE cell growth. Biochem Biophys Res Commun, 1998. 252(2): p. 513-6.

17. Xin, L., et al., Kringle 1 of human hepatocyte growth factor inhibits bovine aortic endothelial cell proliferation stimulated by basic fibroblast growth factor and causes cell apoptosis. Biochem Biophys Res Commun, 2000.277(1): p. 186-90.

18. Chen, C. T., et al., Antiangiogenic gene therapy for cancer via systemic administration of adenoviral vectors expressing secretable endostatin. Hum Gene Ther, 2000. 11(14): p. 1983-96.

The additional references below are also specifically incorporated herein by reference.

Lee T -H, Rhim T, Kim S S. Prothrombin kringle-2 domain has a growth inhibitory activity against basic fibroblast growth factor-stimulated capillary endothelial cells. J Biol Chem 1998; 273(44): 28805-28812.

Sukhatme V P. Kringle 5 causes cell cycle arrest and apoptosis of endothelial cells. Biochem. Biophys. Res. Corn. 1999; 258: 668-673.

Cao Y, Chen A, Seong Soo A A, Richard-Weidong J, Davidson D, Cao Y, Llinas M. Kringle 5 of plasminogen is a novel inhibitor of endothelial cell growth. J Biol Chem 1997; 272(36): 22924-22928.

Sauter B V, Martinet O, Zhang W -J, Mandeli J, Woo SLC. Adenovirus-mediated gene transfer of endostatin in vivo results in high level of transgene expression and inhibition of tumor growth and metastases. Proc. Natl. Acad. Sci. 2000; 97(9): 4802-4807.

Li H, Lu H, Griscelli F, Opolon P, Sun L -Q, Ragot T, Legrand Y, Belin D, Soria J, Soria C, Perricaudet M, Yeh P. Adenovirus-mediated delivery of a uPA/uPAR antagonist suppresses angiogenesis-dependent tumor growth and dissemination in mice. Gene Therapy 1998; 5: 1105-1113.

Dong Z, Yoneda J, Kumar R, Fidler I J. Angiostatin-mediated suppression of cancer metastases by primary neoplasms engineered to produce granulocyte/macrophage colony-stimulating factor. J. Exp. Med. 1998; 188(4): 755-763.

Cao Y, Ji R W, Davidson D, Schaller J, Marti D, Sohndel S, McCance S G, O'Reilly MS, Llinas M, Folkmann J. Kringle domains of human angiostatin. J. Biol. Chem. 1996; 271(56): 29461-29467.

Mukhina S, Stepanova V, Traktouev D, Poliakov A, Beabealashvilly R, Gursky Y, Minashkin M, Shevelev A, Tkachuk V. The chemotactic action of urokinase on smooth muscle cells is dependent on its kringle domain. J. Biol. Chem. 2000; 275(22): 16450-16458.

Fischer K, Lutz V, Wilhelm O, Schmitt M, Graeff H, Heiss P, Nishiguchi T, Harbeck N, Luther T, Magdolen V, Reuning U. Urokinase induces proliferation of human ovarian cancer cells: characterization of structual elements required for growth factor function. FEBS Lett. 1998; 438(1-2): 101-105.

Koopman J L, Slomp J, de Bart A C, Quax P H, Verheijen J H. Mitogenic effects of urokinse on melanoma cells are independent of high affinity bindng to the urokinase receptor. J. Biol. Chem. 1998; 273(50): 33267-33272.

Rabbani S A, Mazar A P, Bemier S M, Haq M, Bolivar I, Henkin J, Goltzman D. Structural requirements for the growth factor activity of the amino-terminal domain of urokinase. J. Biol. Chem. 1992; 267(20): 14151-14156.

1. An abrogen polypeptide with an amino acid sequence consisting of SEQ ID NO.: 1, 3, 5, or 7.

2. The polypeptide of claim 1 in purified form.

3. A nucleic acid consisting of a sequence that encodes the polypeptide of claim 1, optionally containing a sequence encoding a signal sequence or an affinity purification sequence.

4. An expression vector comprising the nucleic acid of claim 3.

5. A cell containing the polypeptide of claim 1 or progeny thereof.

6. A cell containing the nucleic acid of claim 3 or progeny thereof.

7. A purified polypeptide comprising a fragment of a human protein, the fragment consisting essentially of a kringle domain, wherein the polypeptide reduces cell growth induced by bFGF and VEGF.

8. The polypeptide of claim 7, wherein the reduction in cell growth is in endothelial cells.

9. The polypeptide of claim 7, wherein the kringle domain has the amino acid sequence consisting of SEQ ID NO.: 1, 3, 5, or 7.

10. The polypeptide of claim 7, wherein the plasminogen activator is urokinase plasminogen activator.

11. A purified polypeptide of claim 7, consisting of a kringle domain from a human protein, the kringle domain having a region of SEQ ID NO.: 1 from Asn 53 to Asp 59 [NYCRNPD], the polypeptide further having a combination of region selected from the following group: a region of approximately 50% amino acid identity to the region of SEQ ID NO.: 1 from Cys 3 to Trp 27 and a region of approximately 40% amino acid identity to the region of SEQ ID NO.: 1 from Asn 53 to Cys 84; a region of approximately 55% amino acid identity to the region of SEQ ID NO.: 1 from Cys 3 to Trp 27 and a region of approximately 45% amino acid identity to the region of SEQ ID NO.: 1 from Asn 53 to Cys 84; a region of approximately 35% amino acid identity to the region of SEQ ID NO.: 1 from Cys 3 to Trp 27 and a region of approximately 35% amino acid identity to the region of SEQ ID NO.: 1 from Asn 53 to Cys 84; wherein the polypeptide reduces endothelial cell growth induced by bFGF and VEGF.

12. The polypeptide of claim 11, the polypeptide additionally having a signal sequence region.

13. The polypeptide of claim 11, the polypeptide additionally having an affinity purification sequence region.

14. The polypeptide of claim 11, wherein the polypeptide reduces tubule formation in cultured endothelial cells.

15. A purified nucleic acid having a sequence that encodes the polypeptide of claim 11.

16. An expression vector comprising the nucleic acid of claim 15.

17. A cell comprising the vector of claim 16 or progeny thereof.

18. A method for identifying a polypeptide that inhibits endothelial cell proliferation induced by bFGF and VEGF, the method comprising selecting a polypeptide having a single kringle domain from a mammalian protein, the kringle domain comprising amino acid residues Asn 53 to Asp 59 of SEQ ID NO.: 1 [NYCRNPD], the kringle domain also containing 6 Cys residues and 2 Trp residues, introducing the polypeptide to an endothelial cell, and measuring the inhibition of tubule formation induced by bFGF and induced by VEGF as compared to a control.

19. A polypeptide identified by the method of claim 18.

20. An abrogen polypeptide with amino acid sequence of SEQ ID NO.: 1, 3, 5, or 7, wherein 1 to about 5 amino acids outside of the consensus region from Asn 53 to Asp 59 of SEQ ID NO.: 1 [NYCRNPD] are conservatively substituted for.

21. An abrogen polypeptide with amino acid sequence of SEQ ID NO.: 1, 3, 5, or 7 modified to contain 1 to about 15 amino acid changes of substitutions, deletions, or additions, wherein the amino acid changes occur in the amino acids from Asn 28 to His 52, Lys 1 to Thr 2, Ala 85 to Asp 86, wherein the polypeptide inhibits endothelial cell tube formation induced by bFGF and VEGF, and wherein the polypeptide has substantially no smooth muscle cell proliferation or migration inducing activity.

22. The polypeptide of claim 21, wherein the polypeptide contains 1 to about 10 amino acid changes from SEQ ID NO.: 1, 3, 5, or 7.

23. The polypeptide of claim 21, wherein the polypeptide contains 1 to about 5 amino acid changes from SEQ ID NO.: 1, 3, 5, or 7.

24. The polypeptide of claim 21, further comprising 1 to about 5 conservative amino acid substitutions outside of the consensus region from Asn 53 to Asp 59 of SEQ ID NO.: 1 [NYCRNPD].

25. A nucleic acid encoding the polypeptide of one of claim 18 to claim 24.

26. An expression vector comprising the nucleic acid of claim 25.

27. A cell comprising the vector of claim 28 or progeny thereof.

28. A method for treating an angiogenesis related disease or disorder comprising selecting an expression vector for expressing an abrogen polypeptide, inserting an abrogen encoding nucleic acid into the vector, and introducing the vector.

29. A method for treating an angiogenesis related disease or disorder comprising administering the abrogen polypeptide of claim 1.

30. The method of claim 30, wherein the disorder is tumor metastasis.

31. The method of claim 30, wherein the vector is an adenoviral vector, an adeno associated viral vector, or a plasmid vector.

32. The method of claim 30, wherein the abrogen encoding nucleic acid has the sequence of SEQ ID NO.: 2, 4, 6, or 8.

33. The abrogen polypeptide of claim 1, wherein the N-terminus of the abrogen polypeptide is coupled to the signal peptide of interleukin 2.

34. The abrogen polypeptide of claim 33, wherein the abrogen polypeptide is further coupled to a stabilizing molecule at its C-terminus or N-terminus.

35. The abrogen polypeptide of claim 34, wherein the stabilizing molecule is a HSA protein or a IgG2a Fe region.

36. The abrogen polypeptide of claim 34, wherein the C-terminus of the abrogen polypeptide is coupled to the stabilizing molecule via a linker polypeptide.

37. The abrogen polypeptide of claim 36, wherein the linker polypeptide has the sequence as set forth in SEQ ID NO: 9 or 10, or comprises the amino acid sequence ARG-LEU, or ASP-ALA.
